(19) United States
(12) Patent Application Publication
(10) Pub. No.: US 2024/0024255 A1
(43) Pub. Date: Jan. 25, 2024

(54) CURCUMINOID COMPOSITION WITH ENHANCED BIOAVAILABILITY AND METHODS THEREFOR

(71) Applicants: Kodimule Shyam PRASAD, Bangalore (IN); Kuluvar Gouthamchandra, Bangalore (IN); Heggar Venkataramana Sudeep, Bangalore (IN); Siddappa Chandrappa, Bangalore (IN); Amrith Raj, Bangalore (IN); Puttaswamy Naveen, Bangalore (IN)

(72) Inventors: Kodimule Shyam PRASAD, Bangalore (IN); Kuluvar Gouthamchandra, Bangalore (IN); Heggar Venkataramana Sudeep, Bangalore (IN); Siddappa Chandrappa, Bangalore (IN); Amrith Raj, Bangalore (IN); Puttaswamy Naveen, Bangalore (IN)

(21) Appl. No.: 18/225,612

(22) Filed: Jul. 24, 2023

Related U.S. Application Data
(60) Provisional application No. 63/391,792, filed on Jul. 24, 2022.

(51) Int. Cl. A61K 31/12; A61K 36/9066; A61K 9/00; A61P 25/16

(52) U.S. Cl. CPC A61K 31/12; A61K 36/9066; A61K 9/0053; A61P 25/16

(57) ABSTRACT

The invention provides a novel curcuminoid composition and methods for its use and manufacture. The composition can be obtained from an extract from Curcuma longa rhizomes. The formulation of the composition provides enhanced bioavailability and stability of bisdemethoxycurcumin (BDMC) under gastrointestinal conditions. The composition of the invention finds use in methods of promoting health and wellness, and methods of treating neurological disorders and inflammatory disorders.

### CROSS-REFERENCE TO RELATED APPLICATIONS

This application claims the benefit of provisional application No. 63/391,792 filed Jul. 24, 2022, the entire contents of which are incorporated herein by reference for all purposes.

### FIELD OF INVENTION

The invention generally relates to botanical compositions. More particularly, the invention relates to a curcuminoid composition having enhanced bioavailability and stability of polyphenols and methods for using and manufacturing the composition in therapeutic, health maintenance and nutritional applications.

### BACKGROUND OF THE INVENTION

Curcuma longa L. (turmeric) is a culinary spice which also finds use in medicinal preparations. The rhizomes of Curcuma longa L. contain bioactive curcuminoids, including curcumin, demethoxycurcumin (DMC) and bisdemethoxycurcumin (BDMC) (Paramasivam et al. 2009). Conventional turmeric extract preparations contain about 70-75% w/w curcumin, about 17% w/w DMC, and about 3% w/w BDMC. Thus, known turmeric extracts have a much higher curcumin content compared to BDMC.

Clinical experiments have documented the effects of known turmeric extract in some applications (Kalpravidh et al. 2010; Lim et al. 2011; Aditya et al. 2012; Panahi et al. 2014). Other research suggests that curcumin provides turmeric extract with its biological activities (Hewlings and Kalman 2017). However, curcumin is characterized by poor bioavailability and chemical instability which severely limits the biological effects of turmeric extract (Anand et al. 2007; Siviero et al. 2015).

What is needed in the art therefore is a curcuminoid composition having enhanced stability and enhanced bioavailability for use in medicinal and health maintenance applications.

### SUMMARY OF THE INVENTION

The inventor surprisingly discovered a novel curcuminoid composition and its method of use and manufacture. The inventive curcuminoid composition has enhanced stability and bioavailability and can be derived from an extract of turmeric rhizomes. The inventive curcuminoid composition finds use in a variety medicinal and health maintenance applications, such as treating inflammation and inflammatory disorders, treating neurological disorders, providing neuroprotection, promoting cognitive function, and providing antioxidant health benefits. Unlike known curcuminoid compositions, the inventive curcuminoid composition has a proportionately high content of BDMC relative to curcumin. Without being limited to any particular theory or mechanism, providing a curcuminoid composition having a proportionately greater amount of BDMC than curcumin increases the bioavailability of curcumin in the digestive system relative to known curcuminoid compositions, such as turmeric extracts.

In view of the inventor's discovery, it is an object of the invention to provide a curcuminoid composition having enhanced stability and bioavailability, wherein the composition comprises a mixture of curcuminoids that contains an amount of bisdemethoxycurcumin (BDMC) and an amount of curcumin, wherein the amount of BDMC is greater than the amount of curcumin.

In some aspects, the mixture comprises about 75±5% w/w BDMC and about 1.2±0.8% w/w curcumin.

In some aspects, the mixture further comprises DMC.

In some aspects, the mixture comprises 10±5% w/w DMC.

In some aspects, the mixture comprises about 75±5% w/w BDMC, about 10±5% w/w DMC and about 1.2±0.8% w/w curcumin.

In some aspects, the mixture is a turmeric extract.

In some aspects, the mixture is a turmeric rhizome extract.

In some aspects, the composition further comprises an artificial excipient.

In some aspects, the composition is in an administration form selected from a powder, liquid, pill, tablet, pellet, capsule, thin film, solution, spray, syrup, linctus, lozenge, pastille, chewing gum, paste, vapor, suspension, emulsion, ointment, cream, lotion, liniment, gel, drop, topical patch, buccal patch, bead, gummy, gel, sol, injection, and combinations thereof.

In some aspects, the composition is combined with a food, beverage or nutritional supplement.

In some aspects, the composition is enclosed in a container, wherein the container includes instructions for a method of using the composition. The instructions can be printed matter. The instructions can provide information on how to use the composition for at least one of providing neuroprotection, promoting cognitive function, treating a neuronal disorder, treating inflammation, treating an inflammatory disorder, promoting joint health, promoting immune health, promoting joint mobility, promoting heart health and improving memory.

It is a further objective of the invention to provide a method of providing neuroprotection, comprising administering to a subject in need thereof an effective amount of the curcuminoid composition disclosed herein.

Administering the composition can arrest, inhibit, delay or prevent a decline in cognitive function in the subject.

The subject can have, or be at risk of developing, at least one of Alzheimer's disease and Parkinson's disease.

Administering the composition can arrest, delay or inhibit the progression of the symptoms of at least one of Alzheimer's disease and Parkinson's disease.

It is a further objective of the invention to provide a method of promoting cognitive function, comprising administering to a subject in need thereof an effective amount of the curcuminoid composition disclosed herein.

Administering the composition can promote one or more of memory, learning, focus, clarity and mental energy.

The subject can have or be at risk of developing Alzheimer's disease.

In some aspects, the subject has had a stroke, or is at risk of having a stroke.

It is a further objective of the invention to provide a method of treating a neurological disorder, comprising administering to a subject in need thereof an effective amount of the curcuminoid composition disclosed herein.

In some aspects, the neurological disorder is Alzheimer' s disease or Parkinson's disease.

The subject can have, or be at risk of developing, at least one of Alzheimer's disease and Parkinson's disease.

Administering the composition can arrest, inhibit, delay or prevent the progression of the symptoms of at least one of Alzheimer's disease and Parkinson's disease.

It is a further objective of the invention to provide method of treating inflammation, comprising administering to a subject in need thereof an effective amount of the curcuminoid composition disclosed herein.

In some aspects, the subject has an inflammatory disorder.

In performing any of the foregoing methods, the composition can be administered topically or systemically.

In performing any of the foregoing methods, the composition can be administered orally.

### BRIEF DESCRIPTION OF DRAWINGS

FIG. 1 shows the HPLC chromatograms of curcuminoids in an embodiment of the inventive composition.

FIG. 2 is a schematic representation of in vitro gastrointestinal simulation.

FIG. 3 shows the HPLC chromatograms of an embodiment of the inventive composition and control turmeric extract before and after gastrointestinal digestion.

FIG. 4 shows the comparative bioavailability of BDMC in an embodiment of the inventive composition versus curcumin in control turmeric extract.

FIG. 5 shows the lipase inhibition activity of an embodiment of the inventive composition versus control turmeric extract after gastrointestinal digestion.

FIG. 6 shows the oral bioavailability of BDMC in an embodiment of the inventive composition versus curcumin in control turmeric extract.

FIG. 7 shows the nitric oxide scavenging activity of an embodiment of the inventive composition versus control turmeric extract.

FIG. 8A shows the xanthine oxidase inhibition activity of an embodiment of the inventive composition versus control turmeric extract.

FIG. 8B shows the lipoxygenase inhibition activity of an embodiment of the inventive composition versus control turmeric extract.

FIG. 9A shows the cell viability of RAW 264.7 cells upon exposure to different concentrations of an embodiment of the inventive composition versus control turmeric extract.

FIG. 9B shows the effect of an embodiment of the inventive composition versus control turmeric extract on nitric oxide production levels in lipopolysaccharide-induced RAW 264.7 cells.

FIG. 10 shows the effect of an embodiment of the inventive composition versus control curcumin on cytokine production in LPS-stimulated RAW 264.7 macrophages.

FIG. 11 shows the effect of an embodiment of the inventive composition versus control curcumin on the protein expression of iNOS, COX-2 and NFkB-α in LPS stimulated RAW264.7 cells.

FIG. 12A shows the inhibitory effects of an embodiment of the inventive composition against acetylcholinesterase.

FIG. 12B shows the inhibitory effects of an embodiment of the inventive composition against butyrylcholinesterase.

FIG. 13 shows the inhibition kinetics of an embodiment of the inventive composition versus galanthamine on acetylcholinesterase (AChE) activity in presence of different concentrations of substrate.

FIG. 14 shows the kinetic analysis of butyrylcholinesterase inhibition by an embodiment of the inventive composition versus galanthamine.

FIGS. 15A and 15B show the 3D structures of acetylcholinesterase and butyrylcholinesterase retrieved from protein data bank.

FIGS. 16A and 16B show the active sites of recombinant human acetylcholinesterase and human butyrylcholinesterase.

FIG. 17A is a representative image of BDMC binding with the active site of acetylcholinesterase.

FIG. 17B is a representative image of BDMC binding with the active site of butyrylcholinesterase.

FIG. 18 shows the inhibitory effect of an embodiment of the inventive composition on monoamine oxidase B inhibition.

### DEFINITIONS

As used herein, the term “subject” includes warm-blooded animals, preferably mammals, including humans.

As used herein, the phrase “effective amount” includes an amount effective, at dosages and for periods of time necessary, to achieve the desired result in a subject. An effective amount of a composition of the invention, as disclosed herein, may vary according to factors such as the disease state, age, and weight of the subject, and the ability of the composition to elicit a desired response in the subject. Dosage regimens may be adjusted to provide the optimum therapeutic response. An effective amount is also one in which any toxic or detrimental effects (e.g., side effects) of the compound are outweighed by the beneficial effects.

As used herein, the phrase “in need thereof” refers to a subject that requires the therapeutic or health benefit effects for which the composition is administered as a result of a disease, disorder or deficiency in the subject. “In need thereof” can, but does not necessarily, refer to a subject that has been diagnosed with, or determined to be as risk of having or developing, a disease, condition or disorder for which the composition is being administered. “In need thereof” can also refer to a subject that merely desires the potential benefits or effects for which the composition is being administered.

As used herein, the term “about” means the numerical quantity, value or amount that is referenced, or that varies (plus or minus) by up to 5%, up to 10%, up to 15%, or up to 20% of the referenced numerical quantity, value or amount.

As used herein, the terms “inhibit,” “inhibits,” “inhibiting,” “inhibited,” and the like mean slowing, but not completely halting, the progression of, or increase in, the referenced condition or parameter as a result of the effects of the inventive composition relative to control conditions that lack the involvement of the inventive composition.

As used herein, the terms “arrest,” “arrests,” “arresting,” “arrested,” and the like mean halting or maintaining the referenced condition or parameter at a static level as a result of the effects of the inventive composition relative to control conditions that lack the involvement of the inventive composition.

As used herein, the terms “delay,” “delays,” “delaying,” “delayed,” and the like mean forestalling the appearance of the referenced condition or parameter for a period of time as a result of the effects of the inventive composition relative to control conditions that lack the involvement of the inventive composition.

As used herein, the terms “prevent,” “prevents,” “preventing,” “prevented,” and the like mean keeping the referenced condition or parameter from appearing or expressing itself as a result of the effects of the inventive composition relative to control conditions that lack the involvement of the inventive composition.

### DETAILED DESCRIPTION

The inventor surprisingly discovered a curcuminoid composition having greater bioavailability and stability than curcuminoid compositions known in the art, including known turmeric extracts. The invention also provides methods of making and using the composition in a variety of therapeutic and health maintenance applications. Unless dictated otherwise by context, “curcuminoid composition” and “composition,” including their use with modifiers such as “of the invention” and “inventive,” are used interchangeably herein. It will be appreciated that the amounts of curcuminoids set forth in this disclosure refer to the amounts of the curcuminoids by weight, unless dictated otherwise by context.

In some embodiments, the inventive composition comprises a mixture of curcuminoids that includes BDMC and curcumin, wherein the amount of BDMC in the mixture is greater than the amount of curcumin in the mixture. The mixture can comprise about 75±5% w/w BDMC and about 1.2±0.8% w/w curcumin. In some embodiments, the mixture comprises 75±5% w/w BDMC and 1.2±0.8% w/w curcumin.

In some embodiments, the mixture further comprises DMC. The amount of DMC in the mixture can be less than the amount of BDMC, but greater than the amount of curcumin. The mixture can comprise about 10±5% w/w DMC. In some embodiments, the mixture comprises 10±5% w/w DMC. In one non-limiting embodiment, the mixture comprises about 75±5% w/w BDMC, about 10±5% w/w DMC and about 1.2±0.8% w/w curcumin. In yet another non-limiting embodiment, the mixture comprises 75±5% w/w BDMC, 10±5% w/w DMC and 1.2±0.8% w/w curcumin. In other non-limiting embodiments, the mixture comprises BDMC and at least one of DMC and curcumin. The mixture can comprise BDMC and at least one of DMC and curcumin, wherein the amount of BDMC in the mixture is greater than the amount of DMC and/or greater than the amount of curcumin in the mixture. The mixture can comprise about 75±5% w/w BDMC and at least one of about 10±5% w/w DMC and about 1.2±0.8% w/w curcumin. The mixture can comprise 75±5% w/w BDMC and at least one of 10±5% w/w DMC and 1.2±0.8% w/w curcumin.

In some embodiments, the composition comprises a mixture of two or more curcuminoids that are present in a ratio. The mixture can contain BDMC and curcumin in a ratio, wherein the amount of BDMC in the mixture is proportionately greater than the amount of curcumin in the mixture. The mixture can contain BDMC and curcumin in a ratio of about 62.5 parts BDMC and about 1 part curcumin. The mixture can contain BDMC and curcumin in a ratio of 62.5 parts BDMC and 1 part curcumin. The mixture can contain BDMC and at least one of DMC and curcumin in a ratio, wherein the amount of BDMC in the mixture is proportionately greater than the amount of DMC and/or curcumin in the mixture. In one non-limiting embodiment, the mixture contains about 62.5 parts BDMC, about 8.3 parts DMC, and about 1 part curcumin. In another non-limiting embodiment, the mixture contains 62.5 parts BDMC, 8.3 parts DMC, and 1 part curcumin. The mixture can contain BDMC and DMC in a ratio, wherein the amount of BDMC in the mixture is proportionately greater than the amount of DMC in the mixture. The mixture can contain BDMC and DMC in a ratio of about 7.5 parts BDMC and about 1 part DMC. The mixture can contain BDMC and DMC in a ratio of 7.5 parts BDMC and 1 part DMC. The ratios described herein can be determined by weight.

The curcuminoid mixture can be, but is not necessarily, derived from turmeric rhizomes. Thus, the curcuminoid mixture disclosed herein can be a turmeric rhizome extract, in some embodiments. The curcuminoid mixture can be a turmeric rhizome extract comprising BDMC and curcumin, wherein the mixture has a greater amount of BDMC than curcumin. The curcuminoid mixture can be a turmeric rhizome extract comprising BDMC, DMC and curcumin, wherein the mixture has a greater amount of BDMC than curcumin. The curcuminoid mixture can be a turmeric rhizome extract comprising BDMC, DMC and curcumin, wherein the curcuminoid mixture has a greater amount of BDMC than DMC, and a greater amount of DMC than curcumin. The curcuminoid mixture can be a turmeric rhizome extract comprising about 75±5% w/w BDMC, about 10±5% w/w DMC and about 1.2±0.8% w/w curcumin. The curcuminoid mixture can be a turmeric rhizome extract comprising 75±5% w/w BDMC, 10±5% w/w DMC and 1.2±0.8% w/w curcumin. Turmeric rhizomes for making the curcuminoid mixture for use with the inventive composition can be fresh rhizomes, dried rhizomes, partially dried rhizomes, or a combination thereof. The turmeric rhizomes can be powdered.

The curcuminoid mixture can be derived from turmeric rhizomes using any suitable process for collecting curcuminoids from a rhizome. Such processes include, without limitation, solvent extraction, extrusion, or a combination thereof. Suitable solvents for obtaining extracts for obtaining the curcuminoid mixture include, but are not limited to, aqueous solvents, alcohol-based solvents, supercritical fluids, polar organic solvents (such as acetone and methylethyl ketone), or combinations thereof. Non-limiting examples of alcohol-based solvents include, but are not limited to, ethanol, isopropyl alcohol, methanol, and combinations thereof. The supercritical fluid can be, but is not necessarily limited to, carbon dioxide.

In some embodiments, the turmeric rhizome extract for use with the composition is subjected to purification to provide a purified mixture of curcuminoids. The extract can be purified by, for example, chromatography, crystallization, or a combination thereof. Suitable chromatography methods and their devices include, but are not necessarily limited to, gel chromatography (e.g., silica gel chromatography), HPLC or a combination thereof. In some non-limiting embodiments, the turmeric rhizome extract is purified by chromatography, and then further purified through crystallization, such as through the use of an alcohol. Suitable alcohols for the crystallization include, but are not necessarily limited to, ethanol, isopropyl alcohol, methanol, or combinations thereof.

Alternatively, the curcuminoid mixture for use with the inventive composition can be obtained by combining isolated (i.e., purified) curcuminoids. For example, the curcuminoid mixture can be obtained by combining two or more of isolated BDMC, isolated DMC and isolated curcumin. The isolated curcuminoids can be combined to provide a curcuminoid mixture comprising BDMC and curcumin, wherein the mixture has a greater amount of BDMC than curcumin. The isolated curcuminoids can be combined to provide a curcuminoid mixture comprising BDMC, DMC and curcumin, wherein the mixture has a greater amount of BDMC than curcumin. The isolated curcuminoids can be combined to provide a curcuminoid mixture comprising BDMC, DMC and curcumin, wherein the mixture has a greater amount of BDMC than DMC, and a greater amount of DMC than curcumin. The isolated curcuminoids can be combined to provide a curcuminoid mixture comprising about 75±5% w/w BDMC, about 10±5% w/w DMC and about 1.2±0.8% w/w curcumin. The isolated curcuminoids can be combined to provide a curcuminoid mixture comprising 75±5% w/w BDMC, 10±5% w/w DMC and 1.2±0.8% w/w curcumin. The isolated curcuminoids can be combined to provide a curcuminoid mixture having the curcuminoid ratios disclosed herein. The composition can be a turmeric extract, wherein the extract is combined with one or more isolated curcuminoids, including isolated BDMC, isolated DMC and isolated curcumin. The extract can be combined with isolated curcuminoids to achieve the relative amounts of BDMC, DMC and curcumin disclosed herein.

The composition can comprise a curcuminoid mixture in combination with at least one excipient. Excipients for use with the inventive composition can be selected on the basis of compatibility with the curcuminoid mixture and the properties of the desired dosage form. Suitable excipients include, but are not limited to, carriers, binders, fillers, flow aids/glidents, disintegrants, lubricants, stabilizers, surfactants, preservatives, diluents, and the like. The composition can comprise one or more artificial excipients. Suitable excipients include, but are not necessarily limited to, those disclosed in the following references, the entire disclosures of which are incorporated herein by reference for all purposes: Remington: The Science and Practice of Pharmacy, 19^{th} Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999). The composition can further comprise one or more of a sweetener, flavor, vitamin, mineral, sugar, protein, amino acid, and starch. The composition can be combined with beverages, foods, nutritional supplements, and snacks. The beverages, foods, nutritional supplements, and snacks can be dietetic.

The composition of the invention can be used in a variety of therapeutic and health maintenance applications, including, but not limited to, providing neuroprotection, promoting cognitive function, treating neurological disorders, treating inflammation, and treating inflammatory disorders.

In some embodiments, the invention provides a method of providing neuroprotection. The method can be practiced by providing the inventive composition, and administering an effective amount of the composition to a subject in need thereof. Administering the composition can arrest a decline in cognitive function, prevent a decline in cognitive function, inhibit a decline in cognitive function, or delay a decline in cognitive function in the subject. The subject can have Parkinson's disease, or Alzheimer's disease, or be at risk of developing Parkinson's disease or Alzheimer's disease. The subject can be advanced in age and experiencing a normal age-related decline in cognitive function. The subject can be a subject that has experienced one or more ischemic or hemorrhagic strokes. Without being limited to any particular theory or mechanism, the inventive composition provides neuroprotection by inhibiting at least one of acetylcholinesterase (AChE) and butyrylcholinesterase (BuChE) activity in the subject. Thus, in some embodiments, the invention provides a composition as disclosed herein for use as an inhibitor of at least one of acetylcholinesterase (AChE) and butyrylcholinesterase (BuChE) in providing neuroprotection.

In other embodiments, the invention provides a method of promoting cognitive function and/or brain health. The method can be practiced by providing the inventive composition, and administering an effective amount of the composition to a subject in need thereof. Administering the composition can arrest a decline in cognitive function, prevent a decline in cognitive function, inhibit a decline in cognitive function, delay a decline in cognitive function, or improve cognitive function in the subject. The subject can have Parkinson's disease, or Alzheimer's disease, or be at risk of developing Parkinson's disease or Alzheimer's disease. The subject can be advanced in age and experiencing a normal age-related decline in cognitive function. The subject can be a subject that has experienced one or more ischemic or hemorrhagic strokes. Administering the composition can promote one or more of memory, learning, focus, clarity and mental energy. Administering the composition can arrest a decline in, prevent a decline in, inhibit a decline in, or delay a decline in one or more of memory, learning, focus, clarity and mental energy. Without being limited to any particular theory or mechanism, the inventive composition promotes cognitive function and/or brain health by inhibiting at least one of acetylcholinesterase (AChE) and butyrylcholinesterase (BuChE) activity in the subject. Thus, in some embodiments, the invention provides a composition as disclosed herein for use as an inhibitor of at least one of acetylcholinesterase (AChE) and butyrylcholinesterase (BuChE) in promoting cognitive function and/or brain health.

In some embodiments, the invention provides a method of treating a neurological disorder. The method can be practiced by providing the inventive composition, and administering an effective amount of the composition to a subject in need thereof. Administering the composition can arrest the progression of the neurological disorder or its symptoms, inhibit the progression of the neurological disorder or its symptoms, delay the progression of the neurological disorder or its symptoms, or reverse the progression of the neurological disorder or its symptoms. The neurological disorder can be Parkinson's disease, Alzheimer's disease or a neurological injury resulting from ischemic or hemorrhagic stroke. Without being limited to any particular theory or mechanism, the inventive composition treats Alzheimer's disease by inhibiting at least one of acetylcholinesterase (AChE) and butyrylcholinesterase (BuChE) activity in the subject. Thus, in some embodiments, the invention provides a composition as disclosed herein for use as an inhibitor of at least one of acetylcholinesterase (AChE) and butyrylcholinesterase (BuChE) in the treatment of Alzheimer's disease.

Without being limited to any particular theory or mechanism, the inventive composition treats Parkinson's disease by inhibiting monoamine oxidase-B (MAO-B) activity in the subject. Inhibiting monoamine oxidase-B (MAO-B) activity in the subject can inhibit, prevent, or arrest the degeneration of the dopaminergic and non-dopaminergic neurons associated with Parkinson's disease. Thus, in some embodiments, the invention provides a composition as disclosed herein for use as a monoamine oxidase-B (MAO-B) inhibitor in the treatment of Parkinson's disease.

The inventor surprisingly discovered that the composition can also function as an anti-inflammatory agent. Without being limited to any particular theory or mechanism, the composition can function as an anti-inflammatory agent due to the composition's nitric oxide radical scavenging activity, its lipoxygenase enzyme inhibitory activity, its xanthine oxidase inhibitory activity, or combinations thereof. Thus, in some embodiments, the composition's anti-inflammatory effects can be used in a method of promoting at least one of joint health, immune health, heart health, and joint mobility, or in a method of improving memory. Such methods can be practiced by administering an effective amount of the inventive composition to a subject in need of the composition's anti-inflammatory effects.

In other embodiments, the composition's anti-inflammatory effects can be used in a method of treating an inflammatory disorder, wherein an effective amount of the inventive composition is administered to a subject in need thereof.

As used herein, an “inflammatory disorder” is intended to include a disease or disorder characterized by, caused by, resulting from, or becoming affected by inflammation. An inflammatory disorder can be caused by or be associated with biological and pathological processes associated with, for example, the lipoxygenase and xanthine oxidase enzymes. Alternatively, an inflammatory disorder can be caused by or be associated with biological and pathological processes associated with, for example, the expression of one or more of iNOS, COX-2 and NFkB-α.

The method can be used to treat inflammatory disorders including, but not necessarily limited to, asthma (e.g., bronchial asthma), acute and chronic inflammatory disorders such as psoriasis, rheumatoid arthritis, osteoarthritis, psoriatic arthritis, allergic rhinitis, allergies such as uticaria, anaphylaxis, drug sensitivity, food sensitivity, cutaneous inflammation such as dermatitis, eczema, psoriasis, and contact dermatitis, sunburn, spondylarthritis, chronic obstruction pulmonary disease, inflammatory bowel disease (Crohn's disease, ulcerative colitis), ankylosing spondylitis, sepsis, vasculitis, and bursitis, autoimmune diseases such as Lupus, Polymyalgia, Rheumatica, Scleroderma, Wegener's granulomatosis, temporal arteritis, hypertension, diabetes, cryoglobulinemia, multiple sclerosis, transplant rejection, osteoporosis, cancer, including solid tumors (e.g., lung, CNS, colon, kidney, and pancreas), Alzheimer's disease, atherosclerosis, viral (e.g., HIV or influenza) infections, chronic viral (e.g., Epstein-Barr, cytomegalovirus, herpes simplex virus) infection, and ataxia telangiectasia.

The methods disclosed herein can be practiced by administering an effective amount of the inventive composition by any route capable of delivering the biologically active components of the composition to the subject in a manner that permits the components to impart the therapeutic and/or health benefits disclosed herein. The composition can be administered systemically and/or locally. Suitable administration routes for the composition include, but are not limited to, auricular, buccal, conjunctival, cutaneous, dental, endocervical, endosinusal, endotracheal, enteral, epidural, extra-amniotic, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal dental, intracoronary, intracorporus cavernosum, intradermal, intradiscal, intraductal, intraduodenal, intradural, intraepidermal, intraesophageal, intragastric, intravaginal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, oropharyngeal, parentera, percutaneous, periarticular, peridural, perineural, periodontal, rectal, inhalation, retrobulbar, soft tissue, subarachnoid, subconjunctival, subcutaneous, sublingual, submucosal, topical, transdermal, transmucosal, transplacental, transtracheal, transtympanic, ureteral, urethral, vaginal, or combinations thereof. In some preferred embodiments, the composition is administered orally. The composition can be administered by irrigation, drip, infusion, or topically by a dressing, patch, or bandage that is in contact with the composition.

The methods of the invention can be practiced by administering to a subject in need thereof an effective amount of the inventive composition. An effective amount of the composition (i.e., an effective dosage) may range from about 5 to about 200 mg/kg body weight., about 50 to about 200 mg/kg body weight, about 100 to about 200 mg/kg body weight, about 150 to about 200 mg/kg body weight, about 5 to about 300 mg/kg body weight., about 50 to about 300 mg/kg body weight, about 100 to about 300 mg/kg body weight, about 150 to about 300 mg/kg body weight, or about 200 to about 300 mg/kg body weight. The effective amount of the inventive composition can be about 100 mg/kg body weight, about 150 mg/kg body weight, about 200 mg/kg body weight, about 250 mg/kg body weight, or about 3000 mg/kg body weight. In some preferred embodiments, an effective amount of the composition is about 100 to about 200 mg/kg body weight. The skilled artisan will appreciate that certain factors may influence the dosage of the effective amount, including but not limited to the desired outcome, the severity of the disease or disorder, previous treatments and administrations, the general health and/or age of the subject, and other diseases present. Moreover, treatment of a subject with an effective amount of the inventive composition can include a single treatment or administration, or can include a series of treatments or administrations. It will also be appreciated that the effective amount of the composition used for the methods herein may increase or decrease over the course of a particular administration regime.

In some aspects of the invention, an effective amount of the composition is administered as one or more dosage units. As used herein, the phrase “dosage unit” refers to a single unit of an administration form as disclosed herein. For example, a single dosage unit can be a single pill containing the inventive composition, two dosage units can be two pills containing the inventive composition, and so forth. When used in reference to a liquid administration form, a “dosage unit” refers to a volume of the inventive composition that is administered in a single dose. For example, a dosage unit can be 5 ml of a liquid containing the inventive composition that is administered in a single dose, such as orally.

### EXAMPLES

The present invention is described in detail by the following examples. Example 1 describes the method of extraction for the inventive composition resulting in a standardized turmeric extract having higher, stabilized BDMC content. Example 2 illustrates the quantitative analysis of curcuminoids in the extract of the inventive composition by HPLC. Example 3 describes the methods to assess the bioavailability of BDMC and curcumin in the standardized turmeric extract and control turmeric extract, respectively. Example 4 describes the comparative oral bioavailability of BDMC in the inventive composition relative to curcumin in control turmeric extract. Example 5 demonstrates the anti-inflammatory activity of the inventive composition. Example 6 demonstrates the neuroprotective effects of the inventive composition. Example 7 demonstrates the lack of oral toxicity of the inventive composition in rats. References to REVERC3 in the figures refer to the composition obtained according to the process of Example 1.

### Example 1—Preparation of Composition

Powdered turmeric rhizomes (100 kg) were extracted using six bed volumes of ethanol (95-98% v/v) at 65° C. for 2 h in a solvent extractor. The extraction was repeated two times and the combined extract after filtration was evaporated to dryness to yield a curcuminoid-rich crude extract. 10.5 kg of extract mixed with 25 kg silica gel was further subjected to chromatographic separation in a 60-120 mesh column (46×2 cm) containing 100 kg of silica gel, using a mobile phase of ethyl acetate: ethanol. The separation was carried out with a beginning ratio of ethyl acetate (99.0%): ethanol (1.0%) and increasing the polarity thereafter with the increment of 1% each time to collect the fractions. Ten eluted fractions (10 L each) were analysed for the presence of curcuminoids using thin-layer chromatography (TLC) silica gel (Merk-60 F254, 0.25 mm thick) plate. Chloroform:ethanol:glacial acetic acid (94:5:1) was the developing solvent system for TLC. Based on the Rf values, the fractions were pooled and evaporated to dryness. The individual curcuminoids isolated by column chromatography were further purified by recrystallization using ethanol at 7° C. The crystals obtained were separated by filtration. The recrystallized fraction was characterized using HPLC.

### Example 2—Characterization of Curcuminoids

### Reagents

Bisdemethoxycurcumin (BDMC) (98%), demethoxycurcumin (DMC) (98.6%) and curcumin (99.5%) were used as reference standards. All reagents and solvents used were of analytical and HPLC grade.

### Preparation of Standard Solution

Accurately weighed reference standards BDMC, DMC and curcumin were separately taken into 50.0 mL standard volumetric flask and dissolved by using diluent (mobile phase) to obtain a stock concentration of 200, 100 and 170 parts per million (ppm) respectively. Working standard solutions were prepared by diluting the 5.0 mL of standard stock solution into 50.0 mL individual standard volumetric flask and made up with diluent (mobile phase) to get a final concentration of 20, 10 and 17 ppm for BDMC, DMC and curcumin, respectively. The solutions were filtered through 0.2 μ nylon syringe filter and inject the solution.

### Preparation of Sample Solution

The standardized turmeric extract (sample extract) was prepared in acetone to achieve the stock concentration of 300 ppm. 5 mL from the stock solution was diluted with the diluent (mobile phase) to achieve final concentration of 30 ppm. The sample solution was filtered through 0.2 μ nylon syringe filter and injected.

### Total Curcuminoids Analysis

The curcuminoids quantification was performed on a Shimadzu LC2030 C Prominence-i (Japan) system. The system was controlled, and data analysed by LabSolutions software. A separation was carried out in Kinetex C-18 column (100 A°, 150 mm×4.6 mm, 5 μm pore size). The mobile phase consists of isocratic elution with a low-pressure gradient using 1 mg/mL citric acid in water: Tetrahydrofuran (60:40) with a flow rate of 1.0 mL/min and the injection volume of 20 μL. All solutions were degassed and filtered through 0.45 μm pore size filter. The column was maintained at 26° C. throughout analysis, and the wavelength was set to 420 nm. The mobile phase was used as a diluent for assay by HPLC analysis and the total required run time was 20 min.

### Results

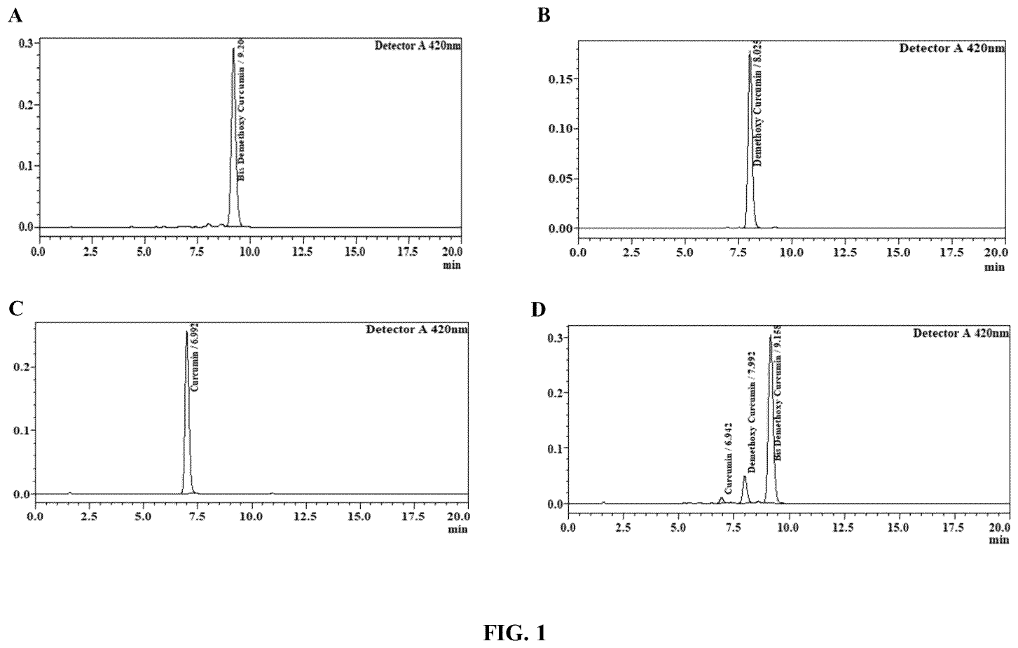

Specificity determined by comparing the chromatogram obtained from blank, standard, and sample solutions are summarized in FIG. 1. The RT of BDMC, DMC and curcumin reference standard and sample peaks were found to be at 9.20, 8.02, 6.99 and 9.15, 7.99, 6.94, respectively. The RT of the standard and sample peak was same so that the method was specific. Good separation between the peaks of BDMC, DMC and curcumin were achieved.

### Example 3—In Vitro Gastrointestinal Digestion

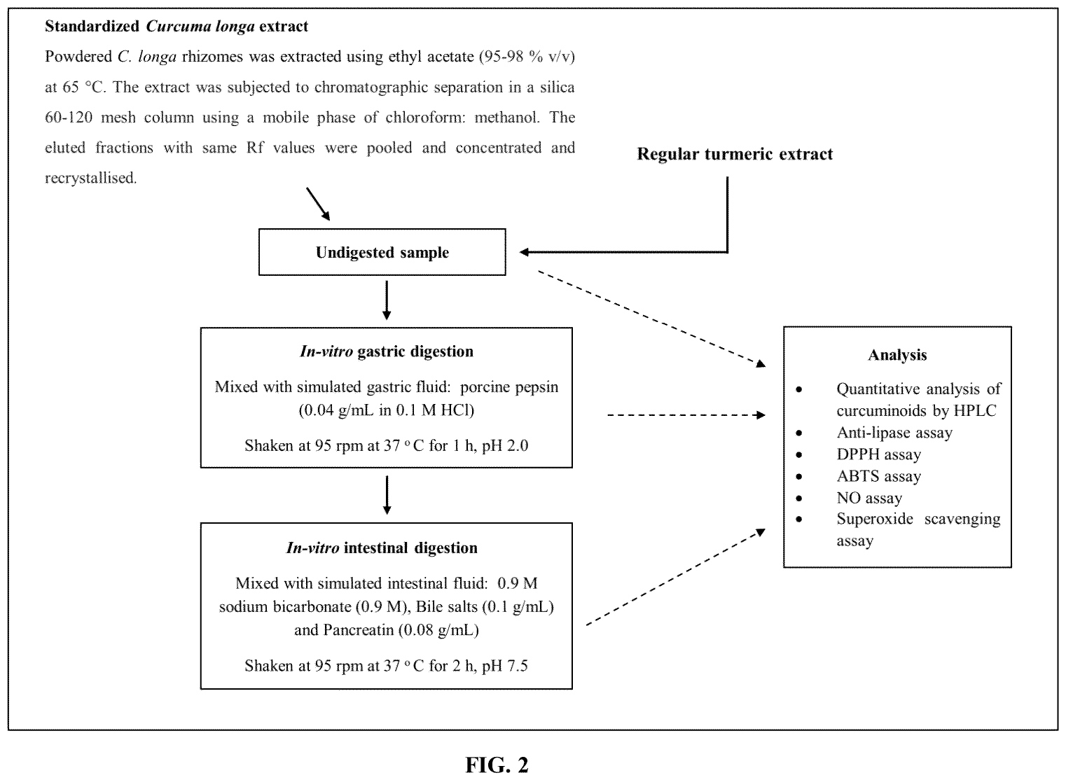

The composition obtained from Example 1 and control turmeric extract were subjected to simulated gastrointestinal digestion described elsewhere with slight modifications (Ryan et al. 2008) (FIG. 2). Briefly, 20 mL of the saline solution containing 1 mL plant extracts were acidified to pH 2.0 by adding porcine pepsin preparation (40 mg/mL in 0.1 M HCl) and allowed for gastric digestion at 37° C. in a shaker incubator. After 1 h, the pH of the solution was adjusted to 5.3 using 0.9 M NaHCO₃ solution. To this added 200 μL of bovine and porcine bile extract solution (100 mg/mL in saline), and 100 μL of pancreatin solution (80 mg/mL in saline). The pH was readjusted to 7.5 using 1 M NaOH followed by incubation at 37° C. for 2 h to accomplish the intestinal digestion phase. Aliquots of samples before and after digestion were centrifuged at 5000 g; upper phase used for anti-lipase and antioxidant assays. The samples were further used for HPLC analysis to determine the bioavailability.

HPLC analysis of the samples before and after gastrointestinal digestion was performed following the in-house validated chromatographic conditions using a Shimadzu LC2030 C Prominence-i (Japan) system equipped with a high sensitivity LC2030 ultraviolet (UV) detector and LabSolutions software. Separation was achieved in Kinetex C-18 column (100 A°, 150 mm×4.6 mm, 5 μm pore size), injecting 10 μL with an autoinjector, using a isocratic elution composed of 0.1% Formic acid:Acetonitrile (50:50) at a flowrate of 1 mL/min. Corresponding standards for BDMC and curcumin were injected to identify the compounds by retention time.

### Pancreatic Lipase Inhibition Assay of the Digested Extracts

The digested extracts were examined for lipase enzyme inhibition following the method of Kim et al. (2012). Briefly, the reaction mixture consisted of 6 μL of lipase solution (pH 6.8) from the enzyme stock solution of 2.5 mg/mL, 169 μL of Tris buffer (pH 7.0) and 20 μL of digested or undigested samples. The mixture was allowed to stand for 15 min at 37° C. with a subsequent addition of 5 μL p-nitrophenylbutyrate (p-NPB) substrate solution (10 mM in dimethyl formamide). The active enzyme hydrolyses p-NPB to nitrophenol which is measured at 405 nm using UV-visible spectrophotometer. The assay was performed in triplicate for each sample. Percentage inhibition of enzyme activity was determined using the formula:

Inhibition %=100−{B−b/A−a×100}

where ‘A’ is the enzyme activity without sample, ‘a’ is the negative control without sample, ‘B’ is the activity with sample, and ‘b’ is the negative control with sample.

### Determination of Antioxidant Activity

The free radical scavenging ability of the extracts before and after gastrointestinal digestion was investigated using the following assays.

### DPPH Free Radical Scavenging Assay

DPPH scavenging activity was done following the method of Soler-Rivas et al. (2000) with some modifications. Briefly, 10 μL of each extract at distinct phases of digestion were mixed with 100 μL of freshly prepared methanolic solution of DPPH (90 μM) and diluted with 190 μL of methanol in a clear 96-well microplate. Trolox was used as the standard and methanol as negative control. After 30 min of incubation in the dark at ambient temperature, the absorbance was measured at 515 nm in a microplate reader. The radical scavenging activity was expressed as Trolox equivalents per gram dry weight (mg/g TE dry weight). The percentage inhibition was calculated using the formula:

DPPH scavenging activity=(Ab−As/Ab)×100

Where Ab is the absorbance of blank; As is the absorbance of sample.

### ABTS·+Radical Cation Scavenging Assay

The ABTS radical scavenging activity of the samples was determined following the method described by Re et al. (1999). The ABTS stock solution was prepared using 7 mM ABTS and 2.45 mM potassium persulfate, incubated in dark for 16 h. The resultant solution was diluted to an absorbance of 0.700 at 734 nm. 10 μL of the undigested and digested extract samples were mixed with 190 μL of ABTS reagent solution and the absorbance was determined at 734 nm. The results were expressed as percentage scavenging activity.

### Nitric Oxide Scavenging Assay

Nitric oxide radical (NO) scavenging was measured with slight modification (Venkatachalam and Muthukrishnan 2012). Briefly, the reaction mixture (3.0 mL) containing sodium nitroprusside (5 mM) in phosphate-buffered saline (pH 7.3), with or without the extract at different phase of digestion, was incubated at 25° C. for 90 min. The nitric oxide radical thus generated interacted with oxygen to produce the nitrite ion, which was assayed at 30-minute intervals by mixing 1.0 mL incubation mixture with an equal amount of Griess reagent. The absorbance of the chromophore (purple azo dye) formed during the diazotization of nitrite ions with sulfanilamide and subsequent coupling with NED was measured at 546 nm.

### Superoxide Radical Scavenging Assay

This assay was based on the reduction of nitro blue tetrazolium (NBT) (Venkatachalam and Muthukrishnan 2012) in the presence of NADH and phenazine methosulfate under aerobic condition. The 3 mL reaction mixture in Tris buffer (0.02 M, pH 8.0) contained 50 μL of 1 M NBT, 150 μL of 1 M NADH with or without sample. The reaction was started by adding 15 μL of 1 M phenazine methosulfate to the mixture and the absorbance change was recorded at 560 nm after 2 minutes. Percent inhibition was calculated against a control without the extract.

### Statistical Analysis

The data were analyzed using GraphPad Prism 9.0. The statistical analysis was performed by ANOVA followed by post hoc test. The values were considered statistically significant at p<0.05.

### Results

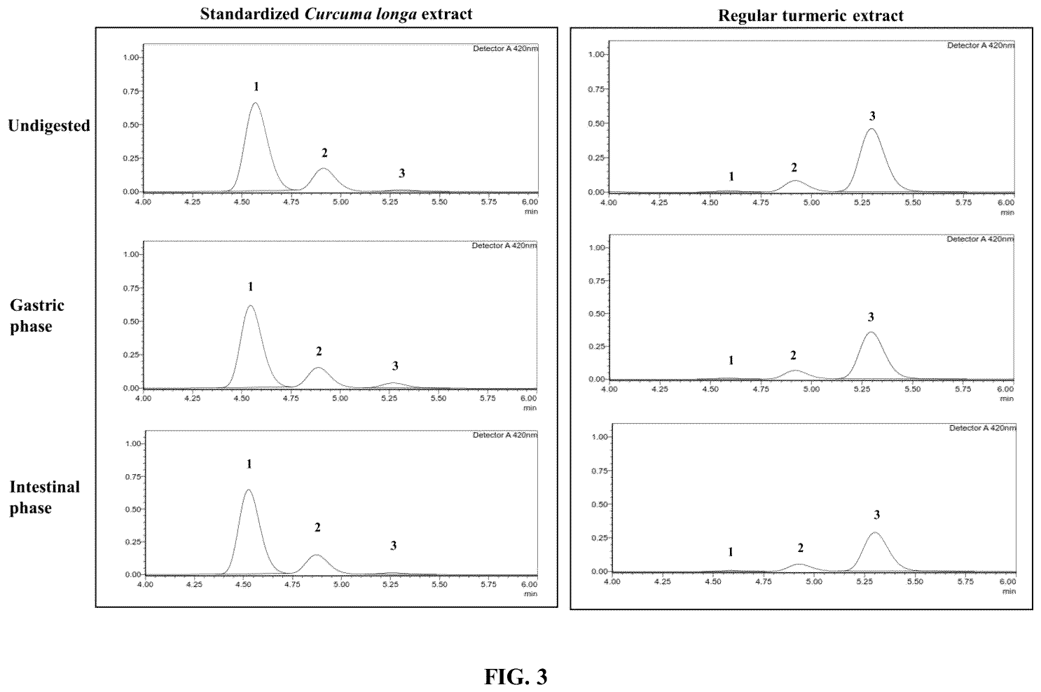

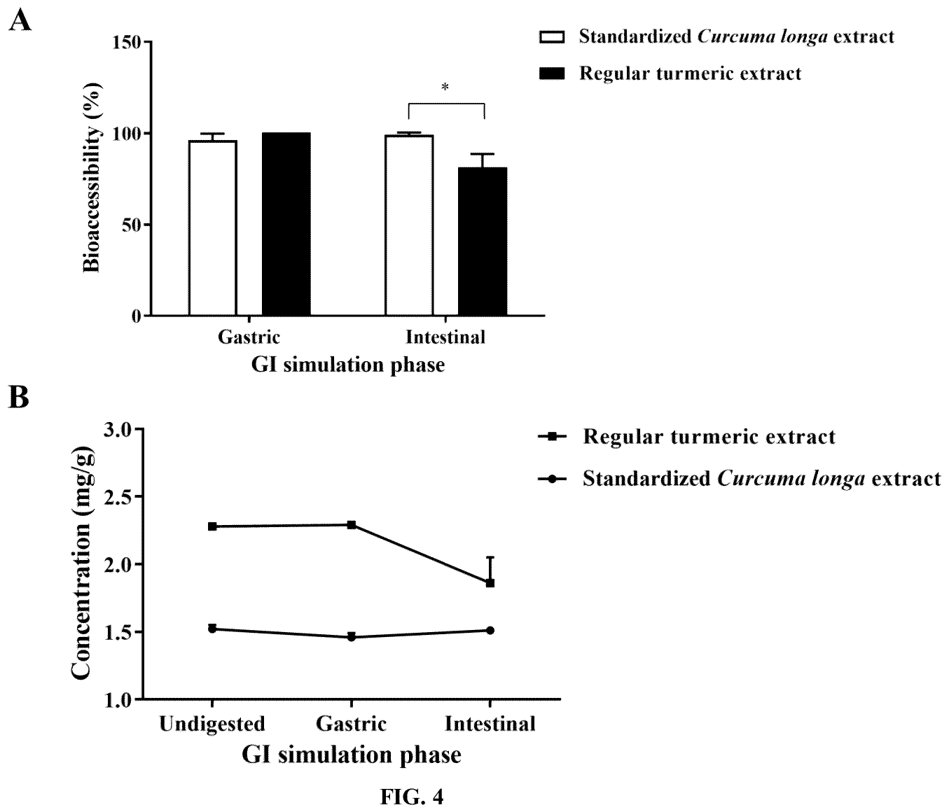

The bioavailability of BDMC (standardized C. longa extract) with respect to undigested sample, was considerably higher (p<0.05) compared to curcumin (RTE). The concentration of BDMC in the samples at different digestive phases remained unchanged. However, the curcumin content in control turmeric extract was markedly reduced in the intestinal phase as compared to the undigested samples (FIGS. 3 & 4).

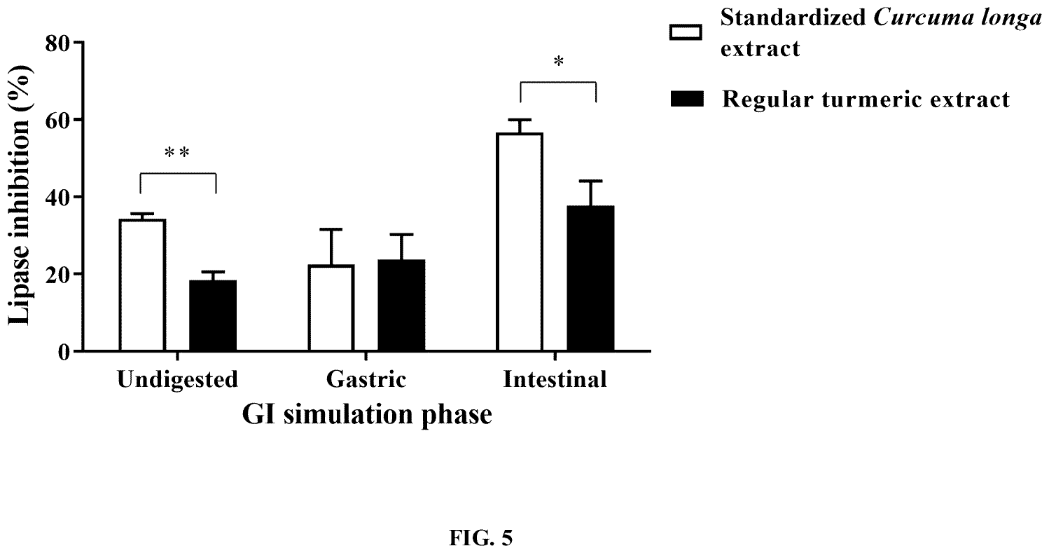

Pancreatic lipase helps to absorb dietary fat in the intestine. It was noted that the in vitro lipase inhibitory activity was markedly increased in both samples (57.17%) and control turmeric extract (45.71%) in the intestinal digestive phase compared to respective undigested samples. However, the lipase inhibitory effect of the sample extract was significantly higher than control turmeric extract (p<0.05). The inhibitory activity of digested sample extract and control turmeric extract were 1.65-fold and 2.05-fold higher than the undigested samples, respectively (FIG. 5).

The radical scavenging activity of turmeric extracts after gastrointestinal digestion are summarized in Table 1.

The DPPH radical scavenging activity of undigested sample extract was 5.8%. The activity was markedly increased in the gastric phase (27.3%) followed by a decline during the intestinal digestion phase (9.3%). A similar trend was observed in control turmeric extract samples. The digested extracts exhibited considerable increase in ABTS cation scavenging activity from undigested to intestinal digestion phase. The percentage inhibition of ABTS radical was significantly higher (p<0.01) in sample extracts (67.36%) as compared to the control turmeric extract (61.58%) in the intestinal digestion phase.

The NO scavenging activity of the sample extract was decreased from 62.2% in the undigested sample to 45.6% after intestinal digestion. The undigested control turmeric extract showed significantly lower NO scavenging activity (25.9%) as compared to sample extract (p<0.001). However, the activity was increased to 64% in the gastric phase followed by a drastic decline (19%) during intestinal digestion. The sample extract exhibited superior NO scavenging activity compared to RTE before and after digestion (p<0.001). The superoxide radical scavenging activity of undigested turmeric extract was markedly increased in the gastric phase while the activity was reduced in the intestinal phase. After gastrointestinal digestion, the sample extract showed significantly higher activity than control turmeric extract (p<0.05).

TABLE 1
Antioxidant activities of the turmeric extracts
before and after gastrointestinal digestion.

| | | | |
|---|---|---|---|
| Digestive phase | Sample Extract | Control Extract | p value# |

DPPH radical scavenging (%)

| | | | |
|---|---|---|---|
| Undigested | 5.77 ± 1.21 | 10.65 ± 0.71 | 0.004** |
| Gastric | 27.33 ± 0.46 | 28.43 ± 1.55 | 0.304 |
| Intestinal | 9.31 ± 1.07 | 11.96 ± 1.91 | 0.104 |

ABTS cation scavenging (%)

| | | | |
|---|---|---|---|
| Undigested | 25.71 ± 1.37 | 23.69 ± 2.47 | 0.283 |
| Gastric | 15.29 ± 1.87 | 14.43 ± 0.60 | 0.492 |
| Intestinal | 67.36 ± 2.20 | 61.58 ± 1.36 | 0.018* |

Nitric oxide scavenging (%)

| | | | |
|---|---|---|---|
| Undigested | 62.20 ± 5.98 | 25.89 ± 3.4 | <0.001* |
| Gastric | 49.11 ± 3.82 | 64.01 ± 4.38 | 0.011* |
| Intestinal | 45.55 ± 3.86 | 18.86 ± 5.24 | 0.002 * |

Superoxide radical scavenging (%)

| | | | |
|---|---|---|---|
| Undigested | 36.98 ± 0.83 | 24.35 ± 0.62 | <0.001** |
| Gastric | 86.47 ± 2.14 | 87.28 ± 0.91 | 0.576 |
| Intestinal | 72.58 ± 0.85 | 68.94 ± 2.17 | 0.054 |

The values are presented as mean ± Standard deviation of three independent experiments. #: Independent t- test. *p < 0.05, **p < 0.01 and ***p < 0.001.

### Example 4—Oral Bioavailability of BDMC

### Animals

Twelve healthy male Wistar rats aged 10-12-week-old (250-300 g) were used for the bioavailability study. The animals were procured from Biogen Laboratory Animal Facility, Bangalore, India (Reg No. 971/PO/RcBiBt/S/06/CPCSEA). All the animals were housed in air-conditioned room under temperature (22±3° C.) and humidity (30-70%) controlled environment. The rats were fed standard rodent diet and water ad libitum. The animal experimentation protocol was approved by the Institutional Animal Ethics Committee (IAEC) of Vidya Herbs Pvt Ltd., Bangalore, India (VHPL/PCL/IAEC/02/2021).

After 7-day acclimatization, the animals were fasted overnight prior to the experiment, with free access to water. Rats were randomized into two groups (n=6): sample and control turmeric extract treatment groups. The sample size was determined by power analysis (Festing and Altman, 2002) using the formula,

Sample size=2SD²(1.96+0.842)²/d² where SD is standard deviation; d is the effect size.

The extracts were formulated in a mixture of Cremaphor, Tween 80, ethanol, and water. The extracts were administered by oral gavage at a single dose of 1000 mg/kg. The animals were anesthetised in anaesthesia chamber supplied with 2% gaseous isoflurane. Blood samples were collected from tail vein at 0.25, 0.5, 1, 2, 4, 8, 12 and 24 h after oral administration of extracts. The blood samples collected in heparinized tubes were centrifuged at 3000 rpm for 10 min. The plasma samples were stored at −20° C. for further analysis. All the animals were rehabilitated after experimentation in accordance with the guidelines laid down by CPCSEA (Committee for the Purpose of Control and Supervision of Experiments on Animals), Government of India.

### Sample Preparation

The plasma samples were extracted using the method described elsewhere with slight modifications (Prasain et al. 2007). Briefly, 50 μL of plasma samples were mixed with 150 μL of methanol and centrifuged at 7000 rpm for 10 min to precipitate the proteins. 100 μL of the supernatant collected and injected 5 μL into the LCMS/MS system.

### Chromatographic Conditions For Quantification of BDMC and Curcumin

LCMS/MS 8050 System (Shimadzu, Japan) equipped with an electrospray ionisation interface (ESI) was used to quantify BDMC and curcumin concentrations in plasma samples. The analysis was conducted on a Kinetex C18 column (150 mm×2.5 mm, 2.6 μm). The mobile phase consisted of 0.2% formic acid and acetonitrile at a flow rate of 0.2 mL/min.

### Statistical Analysis

The data were analyzed using GraphPad Prism 9.0. The statistical analysis was performed by ANOVA followed by post hoc test. The values were considered statistically significant at p<0.05.

### Results

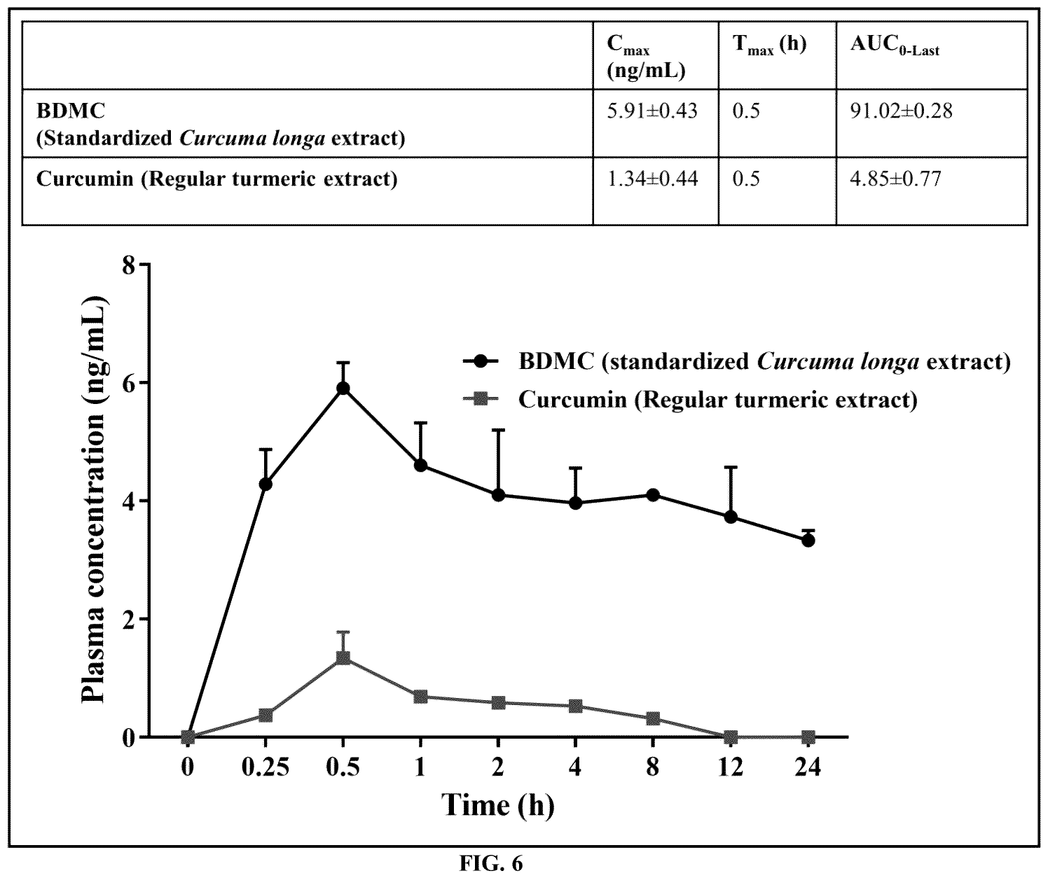

The mean plasma concentration-time of BDMC (standardized C. longa extract) and curcumin (RTE) were compared. The pharmacokinetic measures including Cₘₐₓ, Tₘₐₓ and AUC_{0-Last} are provided in FIG. 6. Plasma levels of curcumin in RTE showed the Cₘₐₓ at 0.5 h, rapidly decreasing thereafter. Interestingly, oral administration of sample extract resulted in a sustained release of BDMC into systemic circulation over 24 h. However, BDMC showed Cₘₐₓ at 0.5 h similar to curcumin. There was a 4.4-fold higher Cₘₐₓ observed for BDMC than curcumin. The AUC_{0-Last} of BDMC was markedly increased in animals administered with sample extract compared to curcumin in control turmeric extract. The relative bioavailability of BDMC was 18.76 compared to curcumin.

### Example 5—Investigation of Antinflammatory Effects

Sample and control turmeric extracts were initially screened for the anti-inflammatory activity using nitric oxide scavenging, lipoxygenase enzyme inhibition and xanthine oxidase inhibition assays. Further the extracts were evaluated for anti-inflammatory effects in lipopolysaccharide (LPS)-induced murine macrophage cells in vitro. The protective effects of sample extract against inflammation was investigated in vivo using carrageenan-induced edema model.

### Nitric Oxide Radical Scavenging

Nitric oxide radical scavenging activity was determined by following method described (Sreejayan and Rao, 1997). Briefly, to 1 mL of different concentrations (5-160 μg/mL) of sample extract and control turmeric extract in phosphate buffer (0.025 M, pH 7.4), 1 mL of sodium nitroprusside (10 mM) was mixed and incubated at 25° C. for 150 min subsequently the 1 mL of Griess reagent (1% sulphanilamide, 2% Orthophosphoric acid and 0.1% N-(1-naphthyl) ethylene diamine) was added and the absorbance was measured at 546 nm. The reaction mixture without the sample was treated as control.

### Lipoxygenase Enzyme (LOX) Inhibition Assay

LOX Inhibition was determined by applying the previously reported method (Kemal et al. 1987). Briefly, sodium phosphate buffer 0.1 M (pH 8.0) and soybean lipoxygenase (165 U/ml) and 10 μL of different concentrations of sample extract and control turmeric extract were combined and incubated at 25° C. for 10 min. 10 μL of 0.32 mM substrate in the form sodium linoleic acid solution was added to initiate the reaction. The formation of (9Z, 11E)-(13S)-13-hydroperoxyoctadeca-9,11-dienoate from sodium linoleic acid by enzymatic conversion was determined by measuring the change in absorbance at 234 nm using UV-Vis spectrophotometer. Negative control was drawn up by replacing samples with sodium phosphate buffer and DMSO into the quartz cuvette. All the reactions were carried out in triplicates.

### Xanthine Oxidase (XO) Inhibition Assay

The inhibition of XO activity was measured spectrophotometrically by adopting the method described by Noro et al. (1983). Briefly, the reaction mixture comprised of enzyme solution (0.04 units/mL in 70 mM phosphate buffer, pH 7.5), and different concentrations of sample, control turmeric extract and 70 mM phosphate buffer (pH 7.5) pre-incubated at 25° C. for 15 min. The reaction was instigated by adding a of a xanthine substrate solution (150 mM). The assay mixture tubes were kept at 25° C. for half an hour and the absorbance was read at 290 nm with UV-Vis spectrophotometer (Shimadzu). The percentage inhibition of XO in the above assay system was calculated using the formula: (1−B/A)×100; where A and B are the activities of the enzyme without and with test material. In this experiment here we define XO as the amount needed to produce 1 mmol of uric acid/min at 25° C.

### Cell Culture

RAW 264.7 cell line (murine macrophage) was purchased from NCCS Pune and cells maintained in 25 cm² tissue culture flask in DMEM added with 100 units/mL penicillin, 100 μg/mL streptomycin and 10% FBS. Cultures were grown at 37° C. in 5% CO₂.

### Cell Viability Assay

Cell viability assay was performed to determine the effect of sample and control turmeric extract on RAW264.7. Briefly, cells (2×10⁴ cells/well) were seeded in 96-well microplates and allowed to adhere overnight and then treated with sample and control turmeric extract separately at varying concentrations 2.5-30 μg/mL, respectively, for 24 h. later, cells were stained with MTT solution for 4 h. The formazan crystals formed were dissolved in 100 μL DMSO and the plates were mildly shaken, the OD was measured using Thermoscan EX reader at 570 nm (Mosmann, 1983).

### Measurement of Nitric Oxide (NO) Level

RAW 264.7 cells were treated with sample and control turmeric extract at two different concentrations (2.5 and 5.0 μg/mL) separately for 4 h, then washed with phosphate-buffered saline, and elicited with LPS (1 μg/mL) for 24 h. Then 100 μL of cell culture supernatant was mixed with 100 μL of Griess reagent. The absorbance (optical density) was read at 540 nm using a microplate reader and NO production was determined using sodium nitrite standard reference curve.

### ELISA Assay

RAW264.7 cells (0.5×10⁵ cells/well) were seeded in 96 well plates and left to adhere overnight. Cells were pre-treated with sample and control extract (2.5 and 5.0 μg/mL) discretely for 1 h, then elicited with LPS (1 μg/mL) for 24 h. The cytokines (TNF-α and IL-6) in the supernatant were measured using an ELISA kit (Krishgen) according to the manufacturer's instructions.

### Western Blot Analysis

Briefly, RAW 264.7 cells were incubated with different concentrations of sample and control turmeric extract separately for 4 h before a 24 h stimulation period with LPS (1 μg/mL). Total protein was extracted from the harvested cells with RIPA buffer, protein concentration was measured using Bradford protein assay, equal amounts of total proteins were separated by SDS-PAGE (8%-12%) and electro transferred to a polyvinylidene fluoride (PVDF) membrane. The membrane was blocked with 5% skim milk for 2 h and subsequently incubated with specific primary antibodies, anti-NOS2 antibody (1:1000, sc-7271), anti-NFκB p65 antibody (1;1000, sc-8008) and anti-Cox-2 antibody (1;1000, sc-19999) for overnight at 4° C. Membranes were later washed three times with Tris-Buffered Saline Tween-20 (TBST) and Tris-Buffered Saline (TBS) incubated further for 2 h with the corresponding secondary antibodies. The blot was visualized using an enhanced chemiluminescence kit.

### In Vivo Anti-Inflammatory Activity

A Grouping of Animals

Wistar rats were randomly allotted into six groups (n=6) and kept for 7-day acclimatization. Group 1 was treated as control, group 2 received indomethacin 10 mg/kg.bw. Rats in groups 3 and 4 were treated with sample extract (100 and 200 mg/kg b.w. p.o.) group 5 and 6 with control turmeric extract (100 and 200 mg/kg.b.w. p.o). All animal studies have been carried out by following the animal ethical guidelines and regulation of Institutional Animal Ethics Committee of Vidya Herbs Pvt. Ltd. Bangalore. India. with the protocol number VHPL/PCL/IAEC/08/19.

B Carrageenan-Induced Acute Inflammatory Model

The carrageenan-induced paw edema test was performed in accordance with the method 17 with some modifications. Drugs were administered to all the respective groups. One hour later, the animals were injected with 0.01 mL of 1% (w/v) freshly prepared carrageenan solution into the right-hind paw sub plantar injection. Paw thickness and volume were measured after 30 min, 1, 2, 4, and 24 h after carrageenan injection using Vernier callipers and Plethysmometer, respectively. The percentage inhibition of inflammation was calculated.

C. Statistical Analysis

Data were analyzed using one-way analysis of variance (ANOVA) with Duncan multiple range test for means ±standard error, where *P<0.001 was considered to indicate statistically significant difference between groups. All the experimental data were derived from three independent experiments.

D. Results

i) Nitric Oxide Scavenging Activity

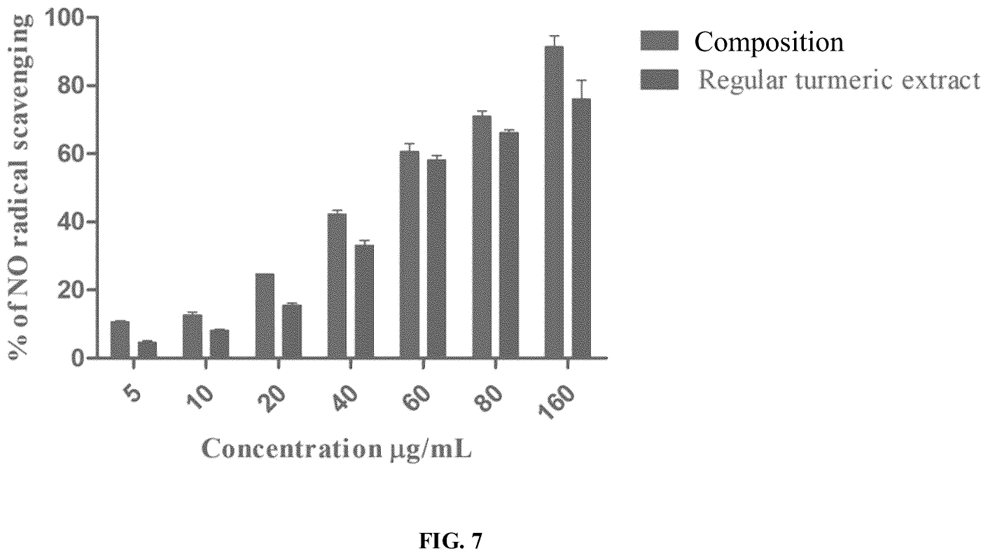

Sample and control turmeric extract inhibited 10-90% at 5-160 μg/mL with an IC50 value 69.5 μg/mL and 79.20 μg/mL (FIG. 7) respectively. Excitingly, sample extract showed strong NO scavenging activity compared to control turmeric extract.

ii) Xanthine Oxidase Inhibition

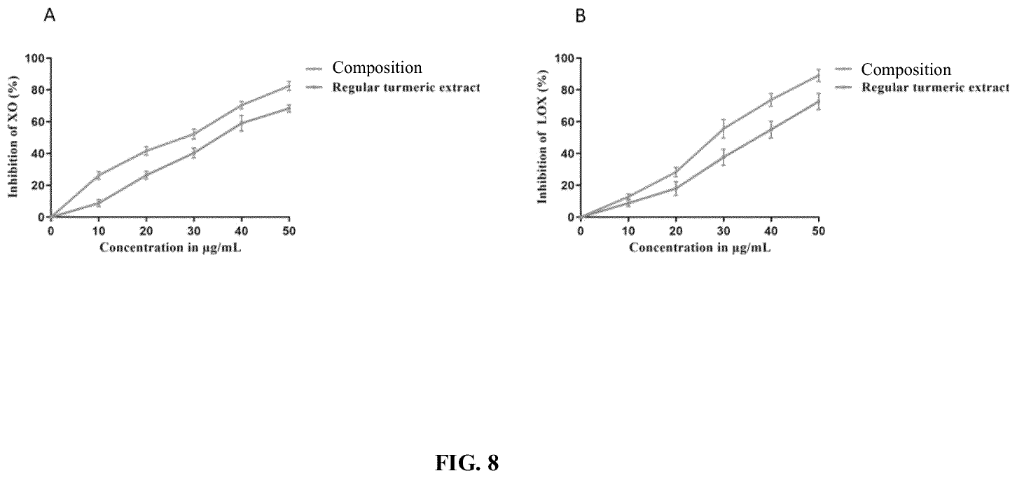

In this study, the level of XO inhibition was evaluated for the sample and control turmeric extract at different concentrations. As illustrated in FIG. 8A, both sample extract and control turmeric extract suppresses the formation of uric acid catalysed by xanthine oxidase significantly in a dose-dependent manner. The concentrations of sample and control turmeric extract causing the half-maximal inhibition (IC50) were determined to be 27.93 μg/mL and 32.83 μg/mL respectively, indicating that sample extract possessed a strong inhibitory activity on XO.

iii) Inhibition of Lipoxygenase (LOX) activity

High concentrations of leukotrienes (LTs) might be observed in the instance of allergic rhinitis, rheumatoid arthritis, asthma, psoriasis, and colitis ulcer which is formed from immune cells. Leukotrienes are a distinctive group of products of the lipoxygenase pathway. Lipoxygenases (LOX) enzymes are linked with allergic and inflammatory reactions. Hence, the LOX inhibition activity is the most important one. As depicted in FIG. 8B sample extract was most effective and inhibited LOX activity up to 89.18% at the concentration of 50 μg/mL with an IC50 value of 24.78 μg/mL whereas control turmeric extract IC50 value was 30.03 μg/mL. Interestingly LOX inhibitory activity of sample extract was strong compared to control turmeric extract.

iv) Effect on RAW 264.7 Cells

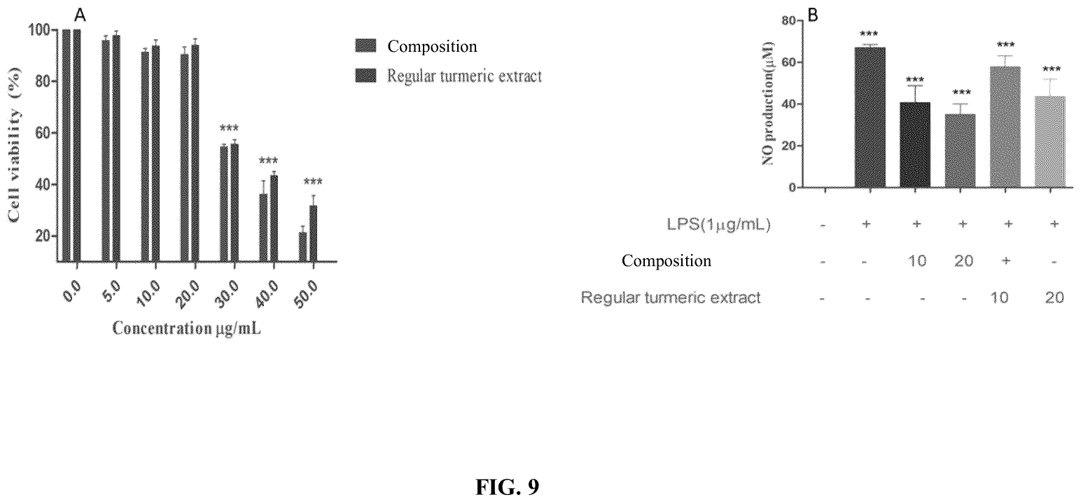

The effect of sample and control turmeric extract on RAW264.7 cell viability was demonstrated by MTT assay. As illustrated in FIG. 9A, our results revealed that sample and control turmeric extract at the concentration of 2.5-7.5 μg did not induce any cytotoxic effect on RAW 264.7 cells, successive experiments were conducted with 2.5 and 5.0 μg/mL of sample and control turmeric extract.

v) Effect on NO Production in LPS-Elicited RAW264.7 Cells

It has been reported that excessive NO is a classical marker for inflammation in LPS stimulated macrophages. As demonstrated in FIG. 9B, LPS induced the dramatic increase of NO in macrophages. Treatment with sample and control turmeric extract significantly lessened the production of NO level. Interestingly, sample extract was better compared to control turmeric extract.

vi) Effect on the Production of Cytokine in LPS-Elicited RAW264.7 Cells

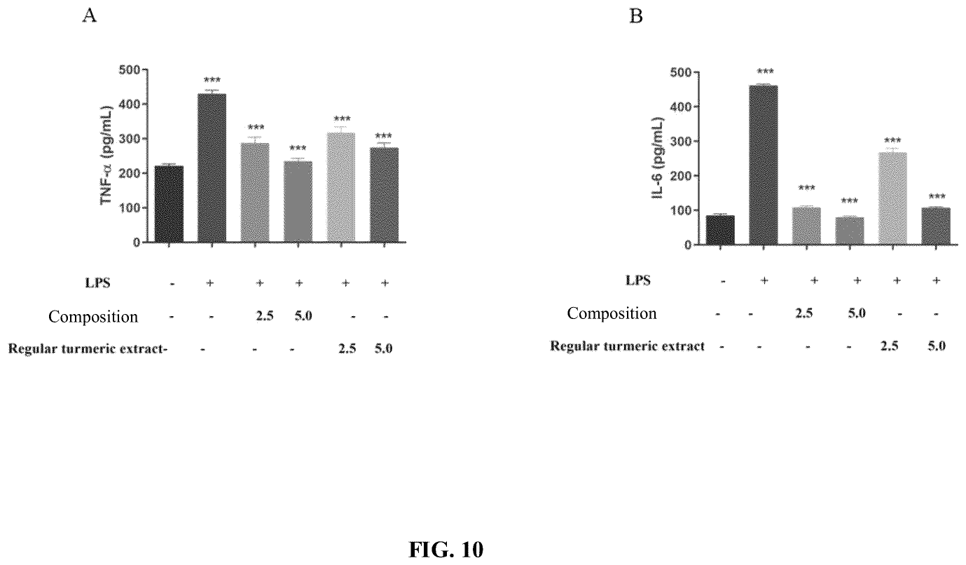

Once inflammation occurs, stimulated macrophages secrete substantial amounts of pro-inflammatory proteins to exacerbate inflammation. As presented in FIGS. 10A and 10B, LPS treatment intensified the expression of TNF-α and IL-6 which could be reduced by the pre-treatment with sample and control turmeric extract. Excitingly sample extract treatment strongly reduced TNF-α and IL-6 cytokine levels compared to control turmeric extract.

vii) Inhibition of iNOS and COX2 Expression in LPS Induced RAW 264.7 Cells

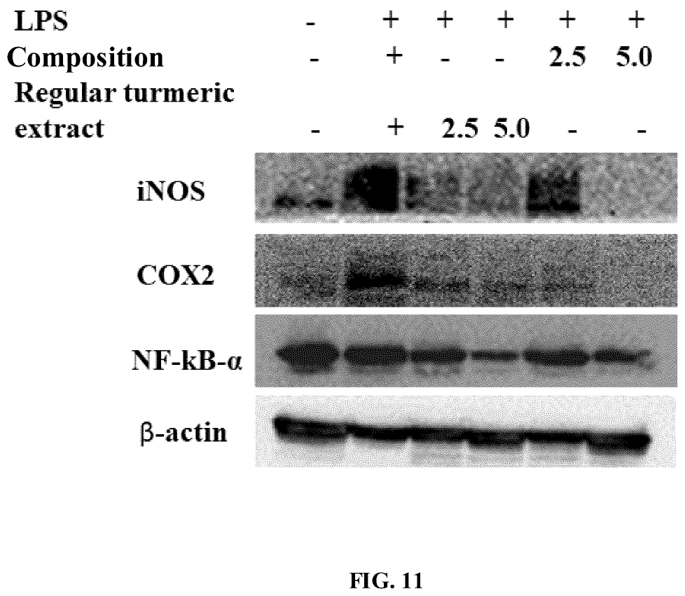

It is reported that COX-2 and iNOS play a significant role in the inflammatory process. Based on the significant inhibition of NO by sample extract, we investigated the expression of COX-2 and iNOS. As shown in FIG. 11, LPS induced the statistically significant expression of COX-2 and iNOS compared to control. Treatment with sample extract and control turmeric extract subsides the expression of COX-2 and iNOS dose-dependently. Sample extract at the concentration of 5.0 μg/mL showed clear inhibition of iNOS and COX2 in comparison to LP S treated cells, whereas control turmeric extract at the concentration of 5.0 μg/mL showed moderate suppression compared to sample extract.

viii) Effect on Carrageenan-Induced Paw

The carrageenan-induced paw edema test was used to assess the anti-inflammatory effects of sample extract (Table 2). After 1 hour of induction, rats treated with sample extract at 100 mg and 200 mg/kg showed significant reduction in paw edema thickness as compared to the untreated vehicle control (p<0.001). Further at the tested doses, sample extract continued to exhibit a significant anti-inflammatory effect up to 24 h. The results were comparable to reference drug indomethacin. Interestingly, sample extract was superior in ameliorating the carrageenan-induced inflammation compared to the control turmeric extract.

TABLE 2
Effect of sample extract on carrageenan-induced paw edema in rats

| | Paw thickness (mm) | | | | | |
|---|---|---|---|---|---|---|
| Treatment | 0 h | 0.5 h | 1 h | 2 h | 4 h | 24 h |
| Vehicle | 2.38 ± 0.16 | 3.70 ± 0.12 | 4.38 ± 0.17 | 4.21 ± 0.12 | 4.11 ± 0.07 | 3.0 ± 0.086 |
| Indomethacin | 2.41 ± 0.12 | 3.18 ± 0.09*** | 3.73 ± 0.07*** | 3.12 ± 0.11*** | 2.67 ± 0.09*** | 2.43 ± 0.10*** |
| Sample | 2.31 ± 0.10 | 3.64 ± 0.18 | 3.97 ± 0.09*** | 3.48 ± 0.30*** | 2.90 ± 0.09*** | 2.43 ± 0.07*** |
| 100 mg/kg | | | | | | |
| Sample | 2.38 ± 0.08 | 3.60 ± 0.15 | 3.88 ± 0.44*** | 3.23 ± 0.18*** | 2.93 ± 0.12*** | 2.44 ± 0.09*** |
| 200 mg/kg | | | | | | |
| TE 100 mg/kg | 2.34 ± 0.11 | 3.53 ± 0.14 | 4.07 ± 0.13** | 3.91 ± 0.08** | 3.18 ± 0.06*** | 2.62 ± 0.19*** |
| TE 200 mg/kg | 2.35 ± 0.11 | 3.44 ± 0.10 | 4.13 ± 0.10* | 3.84 ± 0.18*** | 3.24 ± 0.25*** | 2.56 ± 0.16*** |
| | Paw volume (mL) | | | | | |
| Treatment | 0 h | 0.5 h | 1 h | 2 h | 4 h | 24 h |
| Vehicle | 1.16 ± 0.05 | 1.48 ± 0.03 | 1.76 ± 0.10 | 1.74 ± 0.07 | 1.69 ± 0.06 | 1.38 ± 0.04 |
| Indomethacin | 1.12 ± 0.04 | 1.32 ± 0.04** | 1.57 ± 0.08*** | 1.47 ± 0.06*** | 1.37 ± 0.03*** | 1.16 ± 0.05*** |
| Sample | 1.13 ± 0.04 | 1.37 ± 0.03 | 1.64 ± 0.09* | 1.59 ± 0.11** | 1.43 ± 0.12*** | 1.24 ± 0.07** |
| 100 mg/kg | | | | | | |
| Sample | 1.15 ± 0.06 | 1.37 ± 0.03 | 1.58 ± 0.07*** | 1.47 ± 0.10*** | 1.34 ± 0.07*** | 1.23 ± 0.05** |
| 200 mg/kg | | | | | | |
| TE 100 mg/kg | 1.15 ± 0.08 | 1.40 ± 0.10 | 1.66 ± 0.12 | 1.54 ± 0.11** | 1.39 ± 0.09*** | 1.23 ± 0.07*** |
| TE 200 mg/kg | 1.15 ± 0.06 | 1.39 ± 0.07 | 1.66 ± 0.10 | 1.50 ± 0.07** | 1.38 ± 0.09*** | 1.24 ± 0.08*** |

Values are expressed as mean ± SD (n = 6).
The data were analysed by two way ANOVA followed Bonferroni test.
*p < 0.05, **p < 0.01 and ***p < 0.001 compared to control group.
TE: control turmeric extract

### Example 6—Evaluation of Neuroprotective Effects

### Effect of Sample Extract on Cholinergic Function—Cholinesterase Inhibition

A. Enzyme Inhibition by Ellman Assay

The inhibition of cholinesterase activities was determined using Ellman's assay (Ellman et al. 1961) with modifications. Briefly, 200 μL of the reaction mixture in a 96 well plate contained 5 μL of acetylcholinesterase (AChE) (0.012 U/mL, pH 7.8 sodium phosphate buffer) or butyrylcholinesterase (BuChE) (0.05 U/mL, pH 7.8 sodium phosphate buffer), 100 μL of 5:5-dithiobis-2-nitrobenzoic acid (DTNB) (1.5 mM in pH 7.8 sodium phosphate buffer) and 20 μL of different concentrations of sample extract or galantamine. The reaction mixture was incubated at 25° C. for 10 minutes and then 5 μL of the respective substrate solutions for AchE (0.75 mM acetylthiocholine iodide) or BuchE (0.75 mM butyrylthiocholine iodide) assays were added to initiate the reaction. After 15 min incubation, the absorbance was measured at 405 nm using Ascent Multiskan EX plate reader. The assays were performed in triplicates. The percentage of inhibition was calculated as follows:

% Inhibition=[(A−a)−(B−b)]/(A−a)×100

where, A is the enzyme activity without inhibitor; B is the activity with inhibitor; a and b are the negative controls without and with inhibitor, respectively.

IC50 was determined by nonlinear regression analysis performed using GraphPad Prism version 5.0 (GraphPad Software, San Diego, CA, USA).

B. Kinetic Analysis of Enzyme Inhibition

The kinetics of inhibition of AChE and BuchE activities at various substrate and inhibitor concentrations were studied. Two concentrations of sample extract and galantamine were evaluated for inhibition of enzyme activity using different acetylthiocholine iodide (0.1, 0.2 and 0.4) and butyrylthiocholine iodide (0.1, 0.25, 0.5, 0.75 and 1 mM) substrate concentrations. The changes in reaction velocity were determined as a function of maximum velocity (Vmax) and Michaelis constant (Km). The pattern of inhibition was determined using Lineweaver-Burk (L-B) double reciprocal plot where a graph of 1/change in absorbance (ΔAb/min) was plotted against 1/[substrate]. The identification of the type of inhibition was based on point of intersection of lines. The L-B plots and kinetic parameters Km and Vmax were obtained using GraphPad Prism.

C. Molecular Docking

The crystal structures of recombinant human AChE bound to donepezil (PDB ID: 4EY7, R=2.35 \acute{Å}) and BuChE (PDB ID: 1P0P, R=2.30 \acute{Å}) bound to butyrylthiocholine iodide were downloaded from PDB database (http://www.rcsb.org/) in .pdb format. The coordinates of PDB structures were prepared for molecular docking by removing the water ions and ligands using Python molecule viewer. AutoDock tool (ADT 1.5.4) was used to add polar hydrogens and Gasteiger charges. The 3D structure of BDMC was obtained from Pubchem (https://pubchem.ncbi.nlm.nih.gov). The druggability was determined using SWISSADME prediction (http://www.swissadme.ch/). 3D coordinates were prepared using PRODRG server.

The active site amino acid residues of AChE and BuChE were retrieved from the literature (Stavrakov et al. 2016; Bajda et al. 2013). Molecular docking was performed using AutoDock 4.2. Autogrid was utilized to prepare the grid maps using a grid box size of 50×50×50 xyz points and the active site of AChE (x=20.823, y=16.078, z=18.939) and BuChE (x=137.156, y=113.437, z=43.769). The Lamarckian genetic algorithm and the pseudo-Solis and Wets methods were applied for minimization, using default parameters.

D. Statistical Analysis

IC50 was determined using nonlinear regression analysis and Lineweaver-Burk plots were drawn using linear regression analysis. The analyses were performed, and the graphics generated by GraphPad Prism 9.0.

E. Results

i) Cholinesterase Inhibition

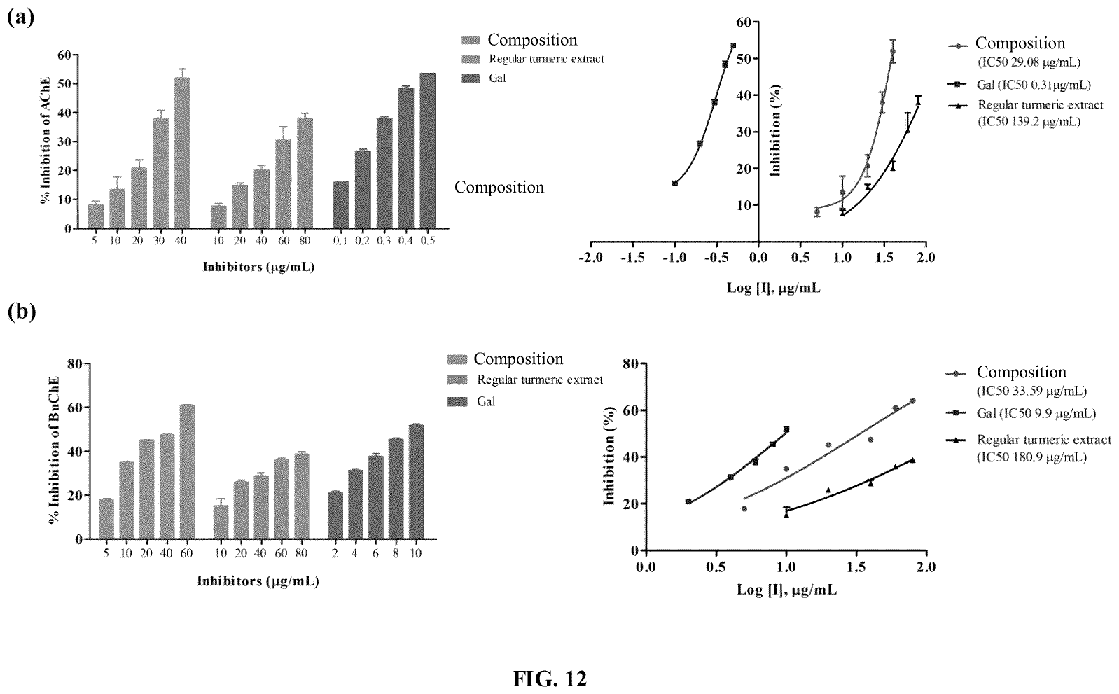

The cholinesterase inhibitory potential of sample extract was determined and compared with control turmeric extract. Galantamine was used as the standard inhibitor. Table 3 and FIG. 12 shows the results of the inhibition assay performed against AChE and BuChE enzymes. As expected, galantamine was far the most potent inhibitor of cholinesterases with IC50 values of 0.31 μg/mL and 9.9 μg/mL for AChE and BuChE activities, respectively. sample extract exhibited higher AchE inhibitory activity (IC50 29.08 μg/mL) compared to control turmeric extract (IC50 139.2 μg/mL). Sample extract and control extract showed 93.8- and 449.03-fold difference relative to galantamine, respectively. A similar trend was observed in BuChE inhibition. Sample extract demonstrated greater potency with an IC50 value of 33.59 μg/mL compared to control turmeric extract (180.9 μg/mL).

TABLE 3
Comparison of IC50 values of inhibitors
against acetylcholinesteraseb (AChE)
and utyrylcholinesterase (BChE)

| | AChE | Fold | | Fold |
|---|---|---|---|---|
| | IC50 | difference | BuChE | difference |
| | value | relative to | IC50 | relative to |
| Inhibitor | (μg/mL) | galantamine | value | galantamine |
| Galantamine | 0.31 | 1 | 9.9 | 1 |
| Sample Extract | 29.08 | 93.8 | 33.59 | 3.39 |
| Control Turmeric | 139.2 | 449.03 | 180.9 | 18.27 |
| Extract | | | | |

ii) Inhibition Kinetics of Cholinesterases

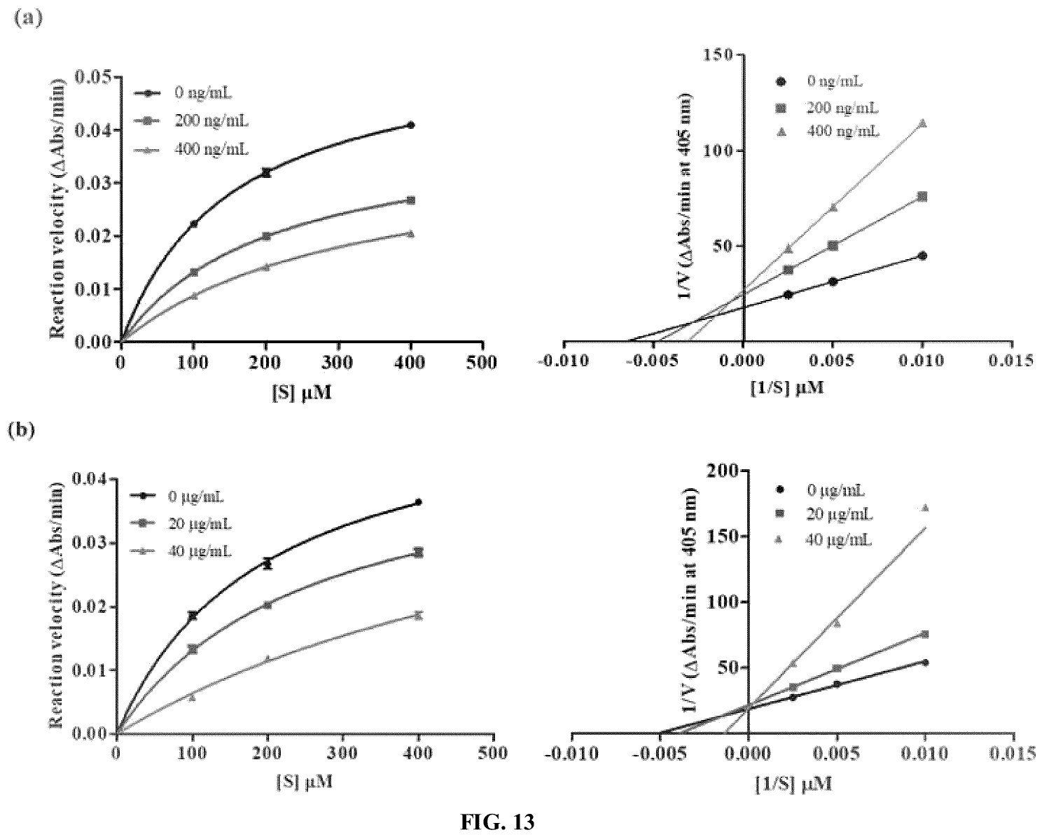

AChE kinetic analysis was performed using different substrate and inhibitor concentrations. FIG. 13A shows the Michaelis-Menten graph and the reciprocal L-B plot of AChE activity in the presence and absence of galantamine. From the data, it appears that galantamine exhibits mixed inhibition of AChE. On the contrary, it was observed from the kinetic analysis that sample extract demonstrated a competitive mode of inhibition (FIG. 13B).

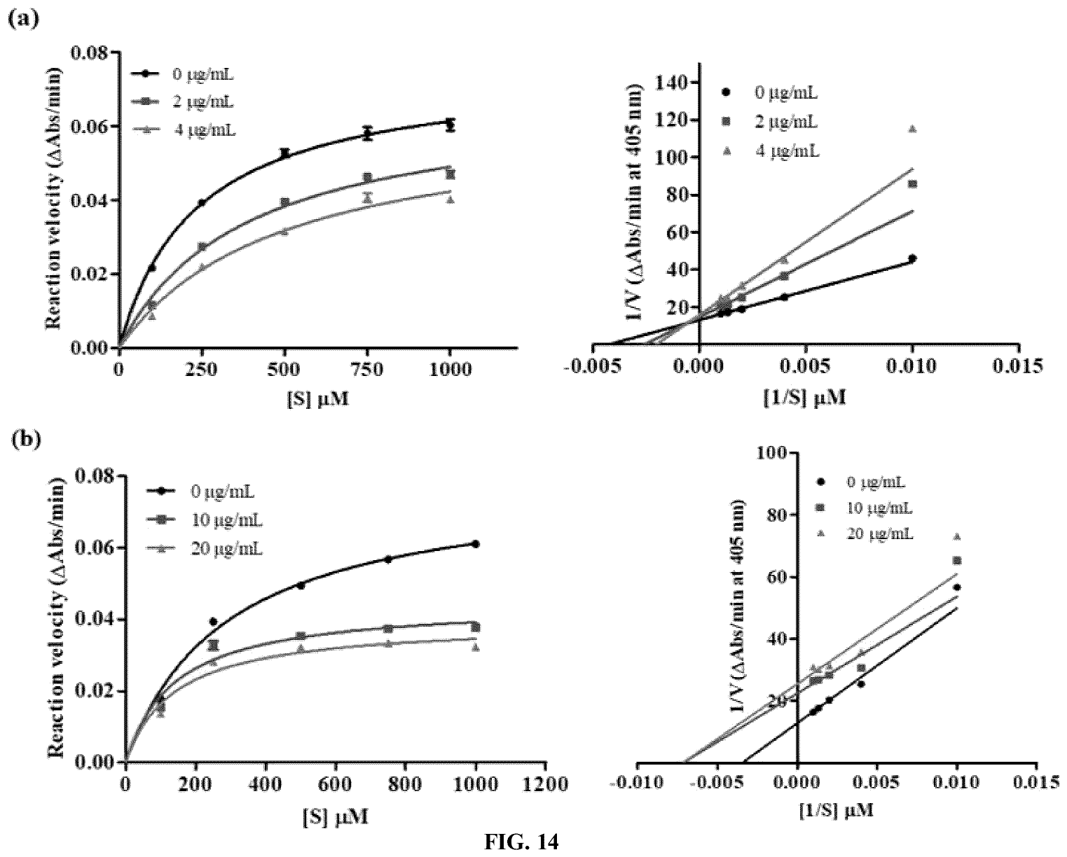

Different concentrations of galantamine and sample extract were further tested for the inhibition of BuChE enzyme, and the kinetic parameters determined. Galantamine was found to inhibit the enzyme activity competitively (FIG. 14A) whereas sample extract exhibited uncompetitive inhibition (FIG. 14B). Sample extract had reduced Vmax (0.04) and Km (139 μM) values compared to the enzyme activity without inhibitor (Vmax 0.08, Km 289.4 μM).

iii) Molecular Docking

BDMC is the major active constituent in sample extract. Here, we have investigated the binding position of BDMC into the active sites of cholinesterases. Initially, SWISSADME was used to predict the drugability of the molecule based on Lipinski's rule of five. We found that BDMC satisfied the drugability criteria (Table 4).

TABLE 4
Drug like properties of BDMC and galantamine

| Lipinski's rule of five | BDMC | Galantamine |
|---|---|---|
| Molecular weight (<500 Da) | 308.33 | 287.35 |
| MLog P (<4.15) | 2.13 | 1.74 |
| H-Bond donor (5) | 2 | 1 |
| H-Bond acceptor (<10) | 4 | 4 |
| Violation | 0 | 0 |

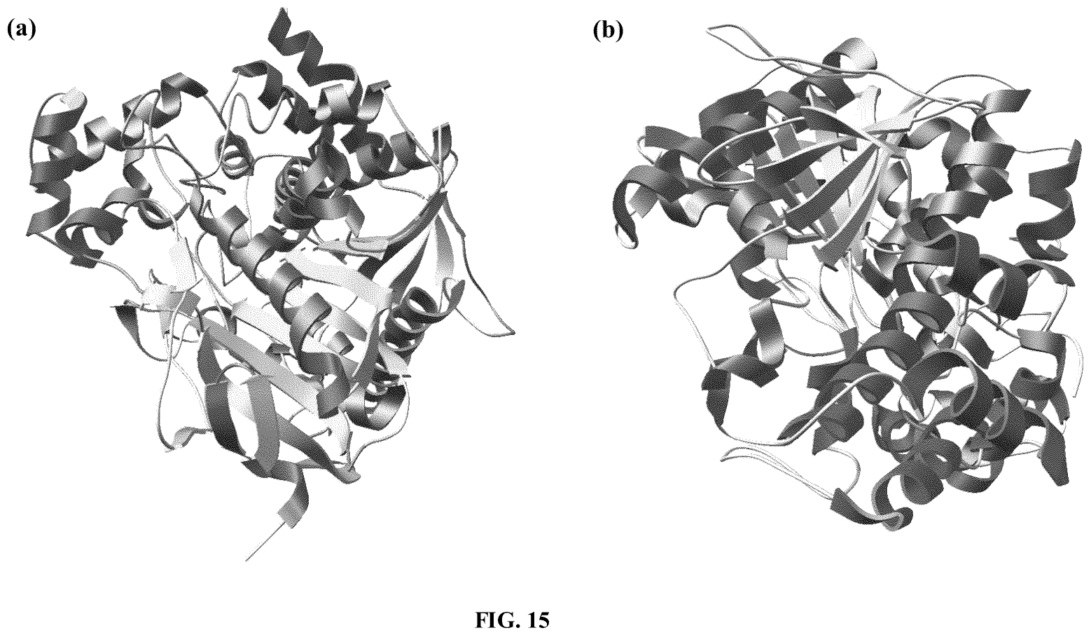

Autodock 4.2 was used to perform the molecular docking analysis. FIG. 15 shows the 3D crystal structures of the cholinesterases.

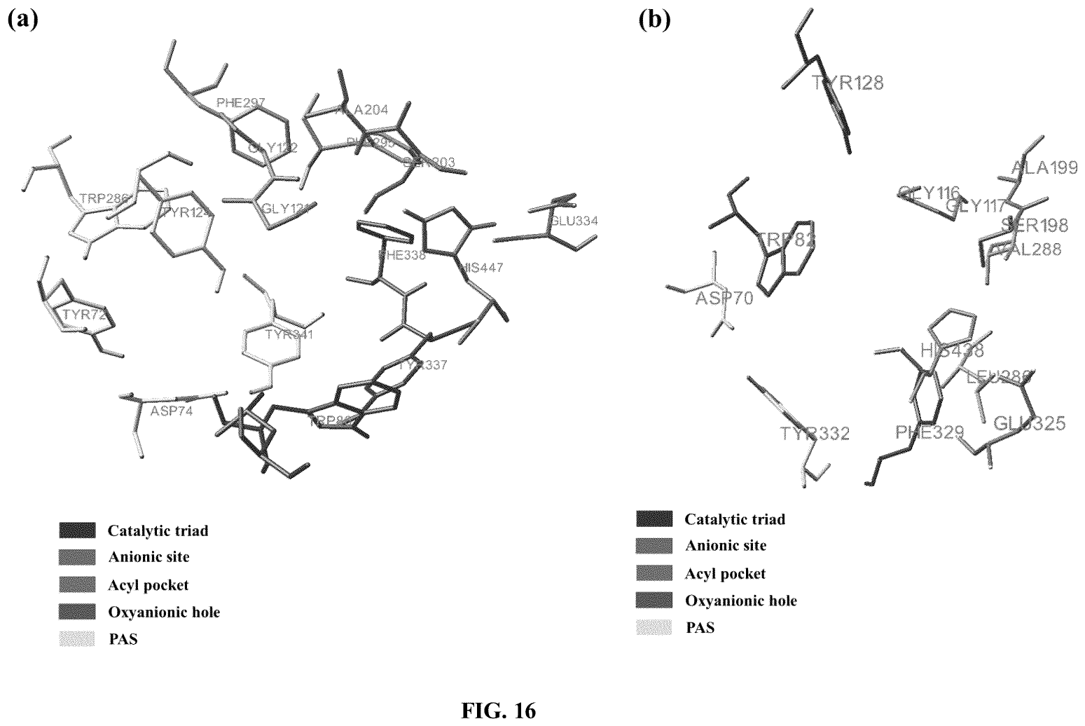

AChE and BuChE enzymes have several domains involved in the substrate binding. In the case of AChE, the catalytic triad is formed by Ser203, Glu334, and His447. The anionic site involved in the binding of choline moiety of ACh contains the aromatic amino acids: Tyr130, Trp86, Tyr337, and Phe338. Another important region in the binding site is the acyl pocket required for the selective binding of ACh (Phe295 and Phe297). Further, the oxyanionic hole formed by Gly121, Gly122, and Ala204 is the site where the structural water molecule stabilizes the enzyme-substrate complex. There exist peripheral anionic site (PAS) in proximity with the catalytic site of the enzyme which allosterically regulates the catalysis. PAS is formed by five residues: Asp74, Tyr72, Tyr124, Trp286, and Tyr341 (FIG. 16A). The human BuChE active site contains binding domains like AChE. However, the small structural differences in the active site are evident due to the difference in several amino acid residues determining the binding domains in the active site (FIG. 16B).

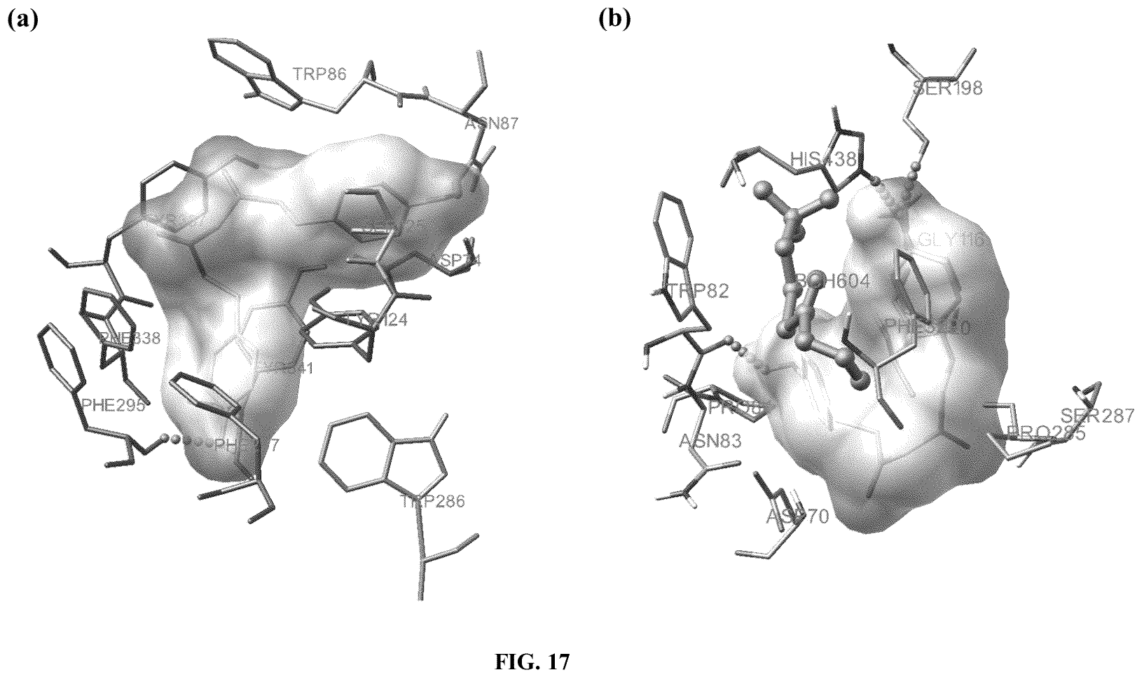

BDMC was docked into the active sites of the cholinesterases and the top ten binding poses were analyzed. The best binding conformation of BDMC with AChE active site showed profound interaction of the molecule with the substrate-binding site of the enzyme. BDMC exhibited hydrogen bond interaction with the key residue Phe295 in the acyl pocket of the active site (FIG. 17A). The lowest binding energy was −7.3 kcal/mol with Ki value of 4.47 μM (Table 5).

TABLE 5
Molecular docking analysis of BDMC
against cholinesterase active sites

| | Binding | | | | VDW-H |
|---|---|---|---|---|---|
| | energy | | Inhibition | Inter- | Bond |
| | (kcal/ | Ligand | constant | molecular | Desolvation |
| Enzyme | mol) | efficiency | (μM) | energy | energy |
| AChE | −7.3 | −0.32 | 4.47 | −10.28 | −10.16 |
| BuChE | −7.27 | −0.32 | 4.73 | −10.25 | −10.09 |

The preference of BDMC for the binding site of BuChE was different as compared to that for AChE (FIG. 17B). Here, we have used the substrate (BTC) bound enzyme as the receptor. BDMC was found to have H-bond interactions with the key residues of the catalytic triad His438 and Ser198. The affinity of BDMC with the enzyme-substrate complex was appreciable (Ki=4.73 μM) with a binding energy of −7.27 kcal/mol.

Overall, the experimental and computational data from this study provides preliminary evidence on the possible role of a BDMC rich turmeric extract in mitigating the cognitive deficits as a function of cholinesterase inhibition. Further, it is important to note that sample extract could act as a dual inhibitor of cholinesterases which substantiates the potential neuroprotective effects of BDMC.

### Evaluation of Monoamine Oxidase-B (MAO-B) Inhibition

Monoamine oxidase-B (MAO-B) is an enzyme that is involved in dopamine metabolism. MAO-B inhibitors have been studied extensively for disease modification in Parkinson's disease (PD). The term disease modification refers to the therapeutic approaches to slow down or modify favorably the degeneration of dopaminergic and non-dopaminergic neurons associated with PD. Drugs that inhibit MAO-B are clinically used to treat neurological disorders and PD. Here, we have evaluated the MAO-B inhibitory effects of sample extract and compared with the control turmeric extract.

A. MAO-B Inhibition Assay

i) Preparation of Rat Liver Homogenate (Enzyme Source)

The rat liver tissues were quickly removed to wash in ice-cold potassium phosphate buffer (0.2 M, pH 7.6), and stored at −80° C. 5 g of liver tissue was homogenized (1:20, w/v) in 0.3 M sucrose at 4° C. and centrifuged at 2000×g for 10 min. The supernatant was further centrifuged at 12000×g for 25 min. The resulting pellet suspended in minimal volume in 0.3 M sucrose and overlay on 1.3 M sucrose and centrifuge at 12000×g (40 min) to obtain a crude mitochondrial pellet. The pellet was resuspended in 2 mL 0.2 M phosphate buffer (pH 7.6) used as MAO-B enzyme source. Total protein concentration was measured by the method of Bradford and adjusted with buffer (0.2 M; pH 7.6) to 0.5 mg protein per ml (stock solution) and stored at −80° C. in aliquots until required.

ii) Assay Procedure

The assay was carried out in 96-well microplates according to the process modified by Holt' et al. (1997). Briefly, 150 μg of enzyme solution, 100 μl chromogenic solution (4 mM vanillic acid (Sigma), 2 mM 4-aminoantipyrine, 8 u/ml peroxidase in potassium phosphate buffer (0.2 M, pH 7.6) and different concentrations of sample extract and control turmeric extract were mixed. The reaction was initiated by adding 70 μL of substrate (tyramine 2 mM), incubated at 37° C. for 90 min. The absorbance was measured in a microplate reader at 450 nm. One unit of activity is defined as the formation of 1 nmole of H₂O₂ per minute per mg protein under the assay condition. The results were plotted in percentage relative activity. Paragyline was used as the standard inhibitor.

iii) Results

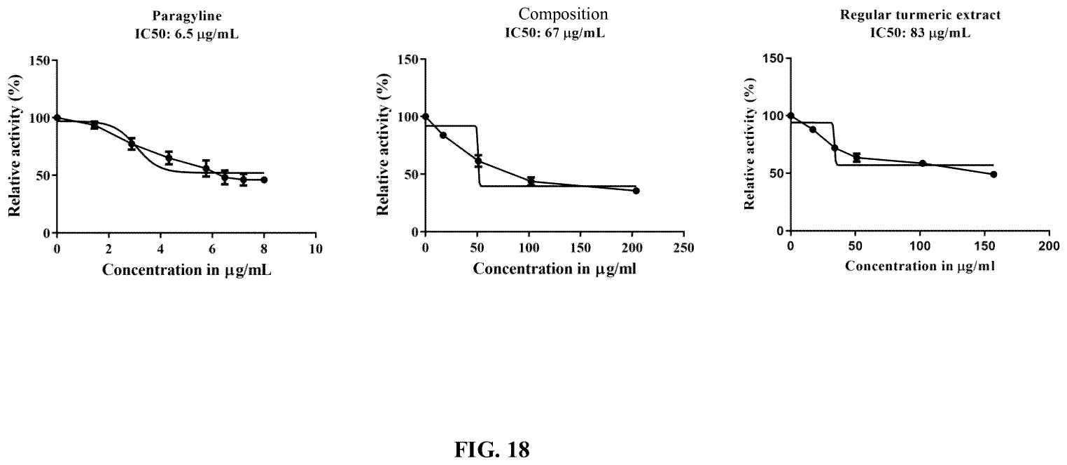

The results of MAO-B inhibitory activity were encouraging wherein the activity of sample extract was found to be higher than the control turmeric extract (FIG. 18). The respective IC-50 values for sample extract and control turmeric extract were 67 μg/mL and 83 μg/mL. The reference standard paragyline showed an IC-50 value of 6.5 μg/mL.

### Example 7—Evaluation of Oral Toxicity

### Acute Oral Toxicity in Wistar Rats

Acute oral toxicity study was conducted to evaluate the toxicity of sample extract upon a single oral administration to Wistar rats followed by observation for 14 days. The study was also intended to identify the LD50 cut-off value of sample extract. The method followed was as per the OECD Guidelines for Testing of Chemicals, Number 423.

Sample extract was initially tested at a dose of 2000 mg/kg b.w. in step 1 with three female rats. Based on survival of previous dosed animals, same dose of 2000 mg/kg b.w. was administered as a confirmatory dose with another set of three females (step 2).

Two steps, consisting of three female Wistar rats each were treated with sample extract by oral gavage administration at a dose of 2000 mg/kg b.w. for step 1 and step 2. The test item was formulated in vehicle (4% Na CMC) at a concentration of 200 mg/mL (step 1 and step 2). The test item was administered at a dose volume of 10 mL/kg b.w.

The animals were observed twice daily throughout the treatment period for mortality and moribundity. The animals were also observed for clinical signs approximately at 30 minutes, 1, 2, 3- and 4- hour on Day 0 post dosing and twice daily during the observation period i.e. Day 1 to Day 14 for each Step. The body weight was recorded on Day 0 (prior to dosing), Day 7 and Day 14. At the end of observation period (Day 14), the animals were euthanized and subjected to a detailed gross pathological examination.

A. Results

No treatment related clinical signs, mortality and moribundity were observed in treated animals throughout the observation period. All the animals gained body weight over the course of the study as compared to day 0 (Table 6). No gross pathological changes were observed in any of the test item treated animals.

TABLE 6
Individual animal body weight and dose administration details

| Step & | | | | Time | | | % Change in Body | |
|---|---|---|---|---|---|---|---|---|
| Dose | | Body | Dose | of | | | weight | |
| (mg/kg | Animal | weight (g) | Volume | Dosing | Body weight (g) | | Day | Day |
| b.w.) | No. | Day 0 | (mL) | (A.M.) | Day 7 | Day 14 | 0-7 | 0-14 |
| Step 1 | 001 | 166.5 | 1.7 | 10:37 | 173.5 | 173.9 | 4.2 | 4.4 |
| & | 002 | 165.5 | 1.7 | 10:38 | 176.6 | 179.2 | 6.7 | 8.3 |
| 2000 | 003 | 160.3 | 1.6 | 10:38 | 174.4 | 176.2 | 8.8 | 9.9 |
| | Mean | 164.10 | — | — | 174.83 | 176.43 | 6.57 | 7.55 |
| | SD | 3.33 | — | — | 1.59 | 2.66 | 2.30 | 2.81 |
| | n | 3 | — | — | 3 | 3 | — | — |
| Step 2 | 004 | 156.6 | 1.6 | 10:27 | 163.7 | 168.8 | 4.5 | 7.8 |
| & | 005 | 159.2 | 1.6 | 10:28 | 174.1 | 182.6 | 9.4 | 14.7 |
| 2000 | 006 | 164.4 | 1.6 | 10:29 | 181.8 | 189.3 | 10.6 | 15.1 |
| | Mean | 160.07 | — | — | 173.20 | 180.23 | 8.16 | 12.55 |
| | SD | 3.97 | — | — | 9.08 | 10.45 | 3.20 | 4.12 |
| | n | 3 | — | — | 3 | 3 | — | — |

SD: Standard deviation;
n: Number of animals

Based on the results of this study, the LD50 cut-off value of sample extract after single oral administration to female Wistar Rats, observed over a period of 14 days is ≥5000 mg/kg b.w. and is classified as ‘Category 5’ or Unclassified based on the Globally Harmonized System of Classification and Labelling of Chemicals (GHS).

B. Repeated Dose Oral Toxicity in Rats

The purpose of this repeated dose toxicity study was to have an approach of the toxic potential of sample extract, when administered orally by gavage to Wistar rat for 28 consecutive days followed by recovery period of 14 days to determine the No-Observed-Adverse-Effect Level (NOAEL).

Total of 60 animals (30 male and 30 female) were assigned to six groups viz., vehicle control (G1), low (G2), mid (G3), high dose (G4), vehicle control recovery (G1R) and high dose recovery (G4R). Each group comprised with 10 animals (5 male and 5 female).

Sample extract was prepared and administered at a dose of 250, 500 and 1000 mg/kg b.w. to G2 (low), G3 (mid) and G4/G4R (high) group animals respectively. The animals in the vehicle control group (G1/G1R) were administered with vehicle (2% Na CMC) alone. The dose volume administered was 10 mL/kg body weight.

The animals were observed twice daily for clinical signs and mortality/moribundity. The detailed clinical examination was carried out for all animals at weekly (±1 day) intervals. The measurement of body weight and feed consumption were done weekly. The ophthalmological and neurological examination was conducted for G1 and G4 group animals at 4th week during treatment period.

At the end of experimental period (day 29 and 43), blood and urine sample were collected and analysed. Subsequently, the animals were sacrificed and subjected to gross pathological examination and the organs were collected, weighed and further G1 and G4 group animal organs were processed for histopathological examination.

C. Results

There were no mortality/moribundity observed in any of the animals of vehicle or test item treated groups throughout the experimental period and no treatment related clinical signs were observed.

No treatment related abnormal neuromuscular and physiological behavioural changes observed in any of the animals of vehicle or test item treated groups (Table 7).

TABLE 7
Summary of functional observational battery

| Parameters | Group & Sex → | | | Gl & | G4 & | G1 & | G4 & |
|---|---|---|---|---|---|---|---|
| | | | | Male | Male | Female | Female |
| | Dose (mg/kg b.w.) → | | | 0 | 1000 | 0 | 1000 |
| | No. of Animals | | | 5 | 5 | 5 | 5 |
| Home cage | Posture | | | 0 | 0 | 0 | 0 |
| | Arousal | | | 0 | 0 | 0 | 0 |
| | Gait | | | 0 | 0 | 0 | 0 |
| Handling | Corneal Reflex | | | 0 | 0 | 0 | 0 |
| | Pinna Reflex | | | 0 | 0 | 0 | 0 |
| | Flexor Reflex | | | 0 | 0 | 0 | 0 |
| | Abdominal Tone | | | 0 | 0 | 0 | 0 |
| | Handling Response | | | 0 | 0 | 0 | 0 |
| | Tail Suspension | | | 0 | 0 | 0 | 0 |
| Open Field Activity | Rearing^{#} | | Mean | 13 | 13 | 11 | 12 |
| | | | ±SD | 2 | 1 | 4 | 3 |
| | Urination^{#} | | Mean | 0 | 0 | 1 | 0 |
| | | | ±SD | 0 | 0 | 1 | 0 |
| | Defecation^{#} | | Mean | 1 | 2 | 1 | 0 |
| | | | ±SD | 1 | 1 | 1 | 0 |
| | Tail Pinch Response | | | 0 | 0 | 0 | 0 |
| | Auditory Startle | | | 0 | 0 | 0 | 0 |
| | Approach Response | | | 0 | 0 | 0 | 0 |
| | Righting Reflex | | | 0 | 0 | 0 | 0 |
| Neuromuscular | Grip strength | Fore limb (gf) | Mean | 613.5 | 548.4 | 555.2 | 615.2 |
| Observation | | | ±SD | 48.8 | 50.1 | 55.6 | 28.2 |
| | | Hind limb (gf) | Mean | 176.0 | 181.1 | 158.8 | 171.1 |
| | | | ±SD | 22.9 | 20.6 | 23.1 | 9.4 |

| | | | | | |
|---|---|---|---|---|---|
| Landing Foot Splay (cm) | Mean | 6.9 | 6.7 | 6.1 | 6.5 |
| | ±SD | 0.5 | 0.6 | 1.2 | 0.9 |
| Motor Activity Measurement | Mean | 1077.2 | 1108.2 | 1361.6 | 1252.8 |
| | ±SD | 226.4 | 55.4 | 215.4 | 240.9 |

0: Normal;
^{#}Values were counted and recorded;
SD: Standard Deviation

No abnormal findings observed in the ophthalmological examination at the end of treatment in both vehicle and high dose group animals.

There were no treatment related effects on body weight, body weight gain (Table 8-10) and food consumption in any of the treatment group animals (Table 11 & 12).

TABLE 8
Summary of body weight and percent change in body weight of male rats

| Group, Sex & | | | | | | | | | | |
|---|---|---|---|---|---|---|---|---|---|---|
| Dose | | Animal Body weight (g) | | | | | Percent (%) Change in Body weight | | | |
| (mg/kg b.w.) | | Day 1 | Day 8 | Day 15 | Day 22 | Day 28 | Day 1-8 | Day 8-15 | Day 15-22 | Day 22-28 |
| G1, M | Mean ± | 151.45 | 176.29 | 203.01 | 225.35 | 235.77 | 17.16 | 15.16 | 11.00 | 4.63 |
| & 0 | SD | 20.47 | 15.48 | 17.91 | 20.20 | 21.81 | 9.27 | 1.01 | 1.99 | 2.49 |
| | n | 5 | 5 | 5 | 5 | 5 | 5 | 5 | 5 | 5 |
| G2, M | Mean ± | 153.60 | 171.57 | 192.90 | 215.99 | 234.80 | 12.12 | 12.75 | 11.99 | 8.65 |
| & 250 | SD | 19.03 | 18.24 | 16.31 | 18.23 | 23.15 | 8.53 | 5.74 | 2.40 | 4.14 |
| | n | 5 | 5 | 5 | 5 | 5 | 5 | 5 | 5 | 5 |
| G3, M | Mean ± | 153.53 | 161.01 | 176.60 | 203.59 | 221.83 | 5.33 | 9.90 | 15.50*↑ | 9.07 |
| & 500 | SD | 22.21 | 17.67 | 19.86 | 18.71 | 18.01 | 5.07 | 8.39 | 2.78 | 3.15 |
| | n | 5 | 5 | 5 | 5 | 5 | 5 | 5 | 5 | 5 |
| G4, M | Mean ± | 149.12 | 169.48 | 183.40 | 205.73 | 225.83 | 14.24 | 7.88 | 12.41 | 9.91 |
| & 1000 | SD | 16.52 | 12.14 | 25.02 | 24.41 | 28.03 | 9.18 | 7.81 | 2.72 | 8.02 |
| | n | 5 | 5 | 5 | 5 | 5 | 5 | 5 | 5 | 5 |

M: Male;
SD: Standard Deviation;
n: No. of Animals;
*p<0.05 vs G1;
↑: Increase

TABLE 9
Summary of body weight and percent change in body weight of female rats

| Group, Sex | | | | | | | | | | |
|---|---|---|---|---|---|---|---|---|---|---|
| & Dose | | Animal Body weight (g) | | | | | Percent (%) Change in Body weight | | | |
| (mg/kg b.w.) | | Day 1 | Day 8 | Day 15 | Day 22 | Day 28 | Day 1-8 | Day 8-15 | Day 15-22 | Day 22-28 |
| G1, | Mean ± | 142.05 | 150.02 | 159.73 | 166.76 | 169.94 | 5.44 | 6.42 | 4.51 | 1.86 |
| F & | SD | 12.26 | 16.59 | 18.77 | 17.95 | 19.27 | 3.15 | 2.13 | 2.64 | 1.17 |
| 0 | n | 5 | 5 | 5 | 5 | 5 | 5 | 5 | 5 | 5 |
| G2, | Mean ± | 143.09 | 154.26 | 166.09 | 175.79 | 179.78 | 7.68 | 7.49 | 5.79 | 2.38 |
| F & | SD | 13.50 | 17.03 | 21.41 | 23.94 | 22.82 | 3.29 | 2.30 | 2.57 | 1.04 |
| 250 | n | 5 | 5 | 5 | 5 | 5 | 5 | 5 | 5 | 5 |
| G3, | Mean ± | 144.12 | 159.45 | 169.02 | 178.94 | 185.01 | 10.63 | 5.81 | 5.72 | 3.51 |
| F & | SD | 11.59 | 15.49 | 20.85 | 25.05 | 24.24 | 6.11 | 4.25 | 3.77 | 1.67 |
| 500 | n | 5 | 5 | 5 | 5 | 5 | 5 | 5 | 5 | 5 |
| G4, | Mean ± | 144.67 | 159.17 | 168.85 | 174.28 | 178.34 | 10.16 | 6.15 | 3.22 | 2.27 |
| F & | SD | 13.37 | 12.60 | 11.70 | 12.56 | 15.09 | 3.29 | 1.47 | 2.48 | 1.93 |
| 1000 | n | 5 | 5 | 5 | 5 | 5 | 5 | 5 | 5 | 5 |

F: Female;
SD: Standard Deviation;
n: No. of Animals

TABLE 10
Summary of body weight and percent change in body weight of recovery groups

| Group, Sex & | | Animal Body weight (g) | | | | | | | Percent (%) Change in Body weight | | | | | |
|---|---|---|---|---|---|---|---|---|---|---|---|---|---|---|
| Dose | | Day | Day | Day | Day | Day | Day | Day | Day | Day | Day | Day | Day | Day |
| (mg/kg b.w.) | | 1 | 8 | 15 | 22 | 29 | 36 | 42 | 1-8 | 8-15 | 15-22 | 22-29 | 29-36 | 36-42 |
| G1R, | Mean ± | 150.29 | 172.37 | 184.96 | 200.28 | 213.01 | 227.36 | 231.34 | 14.75 | 7.24 | 8.46 | 6.49 | 6.80 | 1.76 |
| M & 0 | SD | 15.98 | 18.29 | 21.84 | 21.59 | 19.78 | 20.20 | 20.28 | 3.82 | 3.14 | 4.56 | 2.65 | 2.67 | 0.97 |
| | n | 5 | 5 | 5 | 5 | 5 | 5 | 5 | 5 | 5 | 5 | 5 | 5 | 5 |
| G4R, | Mean ± | 150.59 | 165.83 | 186.38 | 211.26 | 238.47 | 255.40 | 262.60 | 9.88 | 12.78 | 13.51 | 12.68 | 7.05 | 2.80 |
| M & | SD | 15.43 | 21.87 | 20.71 | 21.91 | 33.34 | 36.79 | 39.14 | 3.74 | 7.09 | 4.9 | 7.51 | 0.82 | 1.91 |
| 1000 | n | 5 | 5 | 5 | 5 | 5 | 5 | 5 | 5 | 5 | 5 | 5 | 5 | 5 |
| G1R, F | Mean ± | 141.77 | 153.78 | 161.71 | 171.92 | 177.86 | 182.49 | 186.50 | 8.52 | 5.23 | 6.44 | 3.47 | 2.57 | 2.27 |
| & 0 | SD | 9.23 | 9.96 | 9.23 | 7.00 | 6.80 | 8.83 | 5.81 | 3.88 | 2.89 | 4.00 | 1.24 | 1.75 | 1.98 |
| | n | 5 | 5 | 5 | 5 | 5 | 5 | 5 | 5 | 5 | 5 | 5 | 5 | 5 |
| G4R, | Mean ± | 143.27 | 160.63 | 171.02 | 180.59 | 189.36 | 198.71 | 201.04 | 12.17 | 6.67 | 5.42 | 4.80 | 4.85 | 1.21 |
| F & | SD | 8.77 | 10.63 | 11.89 | 18.26 | 21.17 | 24.40 | 23.88 | 4.81 | 7.72 | 3.63 | 2.37 | 2.46 | 0.52 |
| 1000 | n | 5 | 5 | 5 | 5 | 5 | 5 | 5 | 5 | 5 | 5 | 5 | 5 | 5 |

M: Male;
F: Female;
R: Recovery;
SD: Standard Deviation;
n: No. of Animals

TABLE 11
Summary of feed consumption/animal/day (g) of male rats

| Group, Sex | | | | | | | |
|---|---|---|---|---|---|---|---|
| & Dose | | | | | | | |
| (mg/kg b.w.) | | Week 1 | Week 2 | Week 3 | Week 4 | Week 5 | Week 6 |
| G1, M & | Mean | 15.77 | 17.19 | 18.45 | 19.92 | — | — |
| 0 | SD | 1.78 | 0.65 | 0.01 | 1.31 | — | — |
| | N | 2 | 2 | 2 | 2 | — | — |
| G2, M & | Mean | 14.24 | 16.02 | 17.79 | 18.56 | — | — |
| 250 | SD | 0.06 | 1.06 | 1.37 | 1.20 | — | — |
| | N | 2 | 2 | 2 | 2 | — | — |
| G3, M & | Mean | 15.35 | 14.98 | 16.82 | 18.43 | — | — |
| 500 | SD | 5.34 | 1.45 | 1.82 | 0.87 | — | — |
| | N | 2 | 2 | 2 | 2 | — | — |
| G4, M & | Mean | 15.25 | 15.76 | 18.62 | 17.59 | — | — |
| 1000 | SD | 6.36 | 0.64 | 0.36 | 1.41 | — | — |
| | N | 2 | 2 | 2 | 2 | — | — |
| G1R, M & | Mean | 16.75 | 14.70 | 15.57 | 16.76 | 16.25 | 16.43 |
| 0 | SD | 8.50 | 1.32 | 2.85 | 0.55 | 0.04 | 0.36 |
| | N | 2 | 2 | 2 | 2 | 2 | 2 |
| G4R, M & | Mean | 13.53 | 16.11 | 19.17 | 18.96 | 19.39 | 19.65 |
| 1000 | SD | 0.01 | 0.73 | 1.73 | 1.51 | 2.77 | 1.21 |
| | N | 2 | 2 | 2 | 2 | 2 | 2 |

M: Male;
R: Recovery;
SD: Standard Deviation;
N: No. of cages (each cage housed with 3/2 animals)

TABLE 12
Summary of feed consumption/animal/day (g) of female rats

| Group, Sex | | | | | | | |
|---|---|---|---|---|---|---|---|
| & Dose | | | | | | | |
| (mg/kg b.w.) | | Week 1 | Week 2 | Week 3 | Week 4 | Week 5 | Week 6 |
| G1, F & 0 | Mean | 12.80 | 14.08 | 14.29 | 14.62 | — | — |
| | SD | 2.13 | 2.80 | 2.32 | 3.38 | — | — |
| | N | 2 | 2 | 2 | 2 | — | — |
| G2, F & | Mean | 12.96 | 15.27 | 15.11 | 15.60 | — | — |
| 250 | SD | 2.43 | 2.92 | 3.70 | 3.47 | — | — |
| | N | 2 | 2 | 2 | 2 | — | — |
| G3, F & | Mean | 12.96 | 14.42 | 14.42 | 15.91 | — | — |
| 500 | SD | 2.30 | 3.33 | 2.51 | 2.08 | — | — |
| | N | 2 | 2 | 2 | 2 | — | — |
| G4, F & | Mean | 13.89 | 14.38 | 14.36 | 14.22 | — | — |
| 1000 | SD | 0.78 | 0.99 | 1.34 | 0.89 | — | — |
| | N | 2 | 2 | 2 | 2 | — | — |
| G1R, F & | Mean | 13.05 | 13.96 | 14.23 | 13.81 | 13.25 | 14.05 |
| 0 | SD | 0.50 | 0.24 | 1.01 | 1.01 | 1.14 | 0.19 |
| | N | 2 | 2 | 2 | 2 | 2 | 2 |
| G4R, F & | Mean | 13.61 | 15.02 | 14.83 | 15.12 | 14.94 | 16.05 |
| 1000 | SD | 0.34 | 0.86 | 1.08 | 3.30 | 1.78 | 1.82 |
| | N | 2 | 2 | 2 | 2 | 2 | 2 |

F: Female;
R: Recovery;
SD: Standard Deviation;
N: No. of cages (each cage housed with 3/2 animals)

The haematological (Table 13 and 14), biochemical (Table 15 & 16) and urine analysis (Table 17 & 18) results did not reveal any treatment-related effect in any of the treatment groups.

TABLE 13
Summary of hematology parameters of male rats

| | Group, sex & Dose (mg/kg b.w.) | | | | | |
|---|---|---|---|---|---|---|
| | | | | G4, M & | | G4R, M & |
| Parameter | G1, M & 0 | G2, M & 250 | G3, M & 500 | 1000 | G1R, M & 0 | 1000 |
| BCT (sec) | 44.20 ± 1.48 | 43.40 ± 2.88 | 44.40 ± 3.29 | 44.60 ± 1.14 | 44.80 ± 3.03 | 44.40 ± 2.51 |
| RBC | 6.96 ± 0.48 | 6.65 ± 0.53 | 7.13 ± 0.42 | 7.09 ± 0.46 | 7.08 ± 0.49 | 7.59 ± 0.58 |
| (10¹²/L) | | | | | | |
| HCT (%) | 35.28 ± 2.65 | 33.26 ± 2.67 | 35.84 ± 2.42 | 35.50 ± 2.14 | 34.66 ± 2.79 | 37.44 ± 2.56 |
| PLT (10⁹/L) | 788.00 ± 48.65 | 739.20 ± 143.25 | 628.80 ± 95.99 | 792.80 ± 94.79 | 826.40 ± 140.39 | 638.00 ± 150.23 |
| WBC | 14.34 ± 6.15 | 15.50 ± 4.17 | 13.26 ± 3.75 | 13.44 ± 3.56 | 12.88 ± 3.94 | 12.16 ± 3.60 |
| (10⁹/L) | | | | | | |
| HGB (g/dl) | 12.82 ± 1.00 | 12.10 ± 1.06 | 12.98 ± 0.88 | 12.94 ± 0.74 | 12.62 ± 0.90 | 13.58 ± 0.92 |
| LYM (%) | 74.56 ± 8.18 | 77.98 ± 3.60 | 77.16 ± 2.71 | 70.60 ± 12.62 | 67.40 ± 8.78 | 72.44 ± 4.69 |
| NEU (%) | 20.58 ± 5.30 | 18.34 ± 3.09 | 19.14 ± 2.45 | 25.18 ± 12.27 | 27.42 ± 8.48 | 22.18 ± 3.01 |
| MON (%) | 4.86 ± 3.03 | 3.68 ± 1.37 | 3.70 ± 0.39 | 3.90 ± 2.50 | 4.88 ± 2.95 | 5.38 ± 2.38 |
| EOS (%) | 0.00 ± 0.00 | 0.00 ± 0.00 | 0.00 ± 0.00 | 0.32 ± 0.72 | 0.30 ± 0.67 | 0.00 ± 0.00 |
| BAS (%) | 0.00 ± 0.00 | 0.00 ± 0.00 | 0.00 ± 0.00 | 0.00 ± 0.00 | 0.00 ± 0.00 | 0.00 ± 0.00 |
| RET (%) | 1.96 ± 0.11 | 1.92 ± 0.13 | 2.12 ± 0.24 | 2.18 ± 0.25 | 2.10 ± 0.16 | 2.04 ± 0.11 |
| Bone | Mild increase | Mild increase | NAD/5 | Mild increase | NAD/5 | NAD/5 |
| marrow | in | in | | in | | |
| smear | granulopoietic | granulopoietic | | granulopoietic | | |
| | cells/1, | cells/1, | | cells/1, | | |
| | NAD/4 | NAD/4 | | NAD/4 | | |

M: Male;
SD: Standard Deviation;
R: Recovery;
n: Number of Animals;
BCT: Blood Clotting Time;
Sec: Seconds;
RBC: Total Erythrocyte Count;
HCT: Haematocrit;
PLT: Platelet;
WBC: White Blood Corpuscles;
HGB: Haemoglobin;
LYM: Lymphocytes;
NEU: Neutrophils;
MON: Monocytes;
EOS: Eosinophils;
BAS: Basophils;
RET: Reticulocyte;
NAD: No Abnormality Detected

TABLE 14
Summary of hematology parameters of female rats

| | Group, sex & Dose (mg/kg b.w.) | | | | | |
|---|---|---|---|---|---|---|
| | | | | | | G4R, F & |
| Parameter | G1, F & 0 | G2, F & 250 | G3, F & 500 | G4, F & 1000 | G1R, F & 0 | 1000 |
| BCT (sec) | 43.80 ± 1.48 | 45.60 ± 1.14 | 45.20 ± 1.92 | 45.20 ± 1.92 | 44.40 ± 2.88 | 46.40 ± 2.79 |
| RBC | 6.92 ± 0.28 | 7.06 ± 0.33 | 6.65 ± 0.49 | 6.45 ± 0.43 | 6.95 ± 0.06 | 6.61 ± 0.43 |
| (10¹²/L) | | | | | | |
| HCT (%) | 35.44 ± 0.59 | 36.62 ± 3.29 | 34.16 ± 3.35 | 33.24 ± 1.90 | 33.46 ± 0.87 | 32.36 ± 2.44 |
| PLT (10⁹/L) | 796.20 ± 77.24 | 768.40 ± 68.42 | 704.00 ± 229.66 | 748.60 ± 216.44 | 802.40 ± 15.18 | 766.20 ± 39.78 |
| WBC | 14.26 ± 5.58 | 13.18 ± 5.30 | 8.18 ± 1.60 | 12.38 ± 4.29 | 16.74 ± 4.54 | 10.82 ± 3.54 |
| (10⁹/L) | | | | | | |
| HGB (g/dl) | 12.92 ± 0.34 | 13.46 ± 1.11 | 12.76 ± 1.11 | 12.20 ± 0.75 | 12.52 ± 0.22 | 12.14 ± 0.86 |
| LYM (%) | 76.54 ± 4.23 | 71.16 ± 9.05 | 70.96 ± 7.62 | 79.12 ± 4.19 | 79.20 ± 2.79 | 77.86 ± 3.88 |
| NEU (%) | 19.34 ± 4.31 | 22.60 ± 6.66 | 24.06 ± 5.70 | 17.00 ± 2.35 | 17.78 ± 2.59 | 17.18 ± 2.33 |
| MON (%) | 4.12 ± 1.91 | 6.24 ± 2.53 | 4.98 ± 3.51 | 3.88 ± 2.13 | 2.98 ± 0.38 | 4.96 ± 1.75* |
| EOS (%) | 0.00 ± 0.00 | 0.00 ± 0.00 | 0.00 ± 0.00 | 0.00 ± 0.00 | 0.04 ± 0.09 | 0.00 ± 0.00 |
| BAS (%) | 0.00 ± 0.00 | 0.00 ± 0.00 | 0.00 ± 0.00 | 0.00 ± 0.00 | 0.00 ± 0.00 | 0.00 ± 0.00 |
| RET (%) | 1.94 ± 0.21 | 2.20 ± 0.16 | 1.90 ± 0.12 | 2.04 ± 0.18 | 2.14 ± 0.24 | 2.18 ± 0.18 |
| Bone | Mild increase | Mild increase | Mild increase | Mild increase | NAD/5 | NAD/5 |
| marrow | in | in | in | in | | |
| smear | granulopoietic | granulopoietic | granulopoietic | granulopoietic | | |
| | cells/1, | cells/1, | cells/1, | cells/1, | | |
| | NAD/4 | NAD/4 | NAD/4 | NAD/4 | | |

F: Female;
SD: Standard Deviation;
R: Recovery;
n: Number of Animals;
BCT: Blood Clotting Time;
Sec: Seconds;
RBC: Total Erythrocyte Count;
HCT: Haematocrit;
PLT: Platelet;
WBC: White Blood Corpuscles;
HGB: Haemoglobin;
LYM: Lymphocytes;
NEU: Neutrophils;
MON: Monocytes;
EOS: Eosinophils;
BAS: Basophils;
RET: Reticulocyte;
NAD: No Abnormality Detected;
*p<0.05 vs G1R.

TABLE 15
Summary of clinical chemistry parameters of male rats

| | Group, sex & Dose (mg/kg b.w.) | | | | | |
|---|---|---|---|---|---|---|
| | | | | | | G4R, M & |
| Parameter | G1, M & 0 | G2, M & 250 | G3, M & 500 | G4, M & 1000 | G1R, M & 0 | 1000 |
| ALB (g/dL) | 2.74 ± 0.14 | 2.64 ± 0.26 | 2.61 ± 0.11 | 2.62 ± 0.24 | 2.59 ± 0.08 | 2.57 ± 0.18 |
| ALP (U/L) | 207.40 ± 28.20 | 238.20 ± 47.79 | 300.80 ± 21.25 | 291.00 ± 122.11 | 221.80 ± 22.54 | 248.60 ± 47.45 |
| Ca (mg/dL) | 9.24 ± 0.18 | 8.96 ± 0.25 | 9.24 ± 0.18 | 8.86 ± 0.65 | 10.42 ± 0.30 | 10.70 ± 0.25 |
| CHL (mg/dL) | 63.6 ± 5.32 | 64.4 ± 3.58 | 69.2 ± 10.0 | 73.4 ± 17.3 | 53.2 ± 3.03 | 60.2 ± 7.69 |
| CRE (mg/dL) | 0.52 ± 0.00 | 0.52 ± 0.03 | 0.50 ± 0.05 | 0.53 ± 0.05 | 0.53 ± 0.03 | 0.54 ± 0.03 |
| GLU (mg/dL) | 112.14 ± 16.38 | 116.66 ± 17.99 | 117.74 ± 4.24 | 120.66 ± 12.87 | 103.84 ± 12.52 | 125.54 ± 19.00 |
| P (mg/dL) | 7.82 ± 0.22 | 8.68 ± 0.91 | 8.56 ± 0.24 | 10.00 ± 0.54** | 6.27 ± 0.52 | 6.36 ± 1.00 |
| TP (mg/dl) | 7.07 ± 0.20 | 7.07 ± 0.27 | 7.12 ± 0.07 | 7.16 ± 0.07 | 7.06 ± 0.33 | 7.08 ± 0.37 |
| TRG (mg/dL) | 47.98 ± 12.59 | 56.28 ± 5.44 | 49.88 ± 5.54 | 52.08 ± 12.61 | 49.70 ± 4.51 | 66.10 ± 22.05 |
| Urea (mg/dL) | 44.66 ± 4.97 | 46.96 ± 8.67 | 41.16 ± 7.42 | 45.34 ± 6.01 | 44.18 ± 3.14 | 42.36 ± 6.90 |
| AST (U/L) | 188.46 ± 27.19 | 201.52 ± 18.62 | 163.04 ± 9.67 | 161.38 ± 22.30 | 162.74 ± 13.04 | 155.98 ± 12.06 |
| TBL (mg/dL) | 0.09 ± 0.01 | 0.09 ± 0.01 | 0.08 ± 0.00 | 0.09 ± 0.01 | 0.09 ± 0.01 | 0.10 ± 0.01 |
| ALT (U/L) | 44.36 ± 2.61 | 60.40 ± 18.51* | 47.96 ± 1.96 | 46.36 ± 3.16 | 47.44 ± 14.42 | 41.38 ± 17.79 |
| Na (mmol/L) | 142.64 ± 1.13 | 142.92 ± 1.11 | 143.36 ± 0.35 | 143.56 ± 0.80 | 147.08 ± 1.60 | 146.80 ± 1.15 |
| K (mmol/L) | 5.67 ± 0.28 | 5.79 ± 0.14 | 5.85 ± 0.12 | 5.95 ± 0.17 | 6.29 ± 0.23 | 5.88 ± 0.15# |

M: Male;
SD: Standard Deviation;
R: Recovery;
n: Number of Animals;
ALB: Albumin;
ALP: Alkaline Phosphatase;
Ca: Calcium;
CHL: Cholesterol;
CRE: Creatinine;
GLU: Glucose;
P: Phosphorus;
TP: Total Protein;
TRG: Triglycerides;
AST: Aspartate Aminotransferase;
TBL: Total Bilirubin;
ALT: Alanine Aminotransferase;
Na: Sodium;
K: Potassium;
*p<0.05 and **p<0.001 vs G1;
#p<0.05 vs G1R.

TABLE 16
Summary of clinical chemistry parameters of female rats

| | Group, sex & Dose (mg/kg b.w.) | | | | | |
|---|---|---|---|---|---|---|
| | | | | | | G4R, F & |
| Parameter | G1, F & 0 | G2, F & 250 | G3, F & 500 | G4, F & 1000 | G1R, F & 0 | 1000 |
| ALB (g/dL) | 2.72 ± 0.16 | 2.84 ± 0.28 | 3.04 ± 0.11* | 2.88 ± 0.07 | 2.79 ± 0.11 | 2.82 ± 0.11 |
| ALP (U/L) | 191.40 ± 44.79 | 193.00 ± 58.54 | 169.20 ± 24.20 | 197.80 ± 58.07 | 161.20 ± 42.26 | 162.00 ± 37.48 |
| Ca (mg/dL) | 9.68 ± 0.18 | 9.82 ± 0.28 | 9.80 ± 0.20 | 9.72 ± 0.32 | 10.58 ± 0.29 | 10.84 ± 0.30 |
| CHL (mg/dL) | 71.2 ± 13.5 | 80.8 ± 4.71 | 84.80 ± 9.91 | 80.4 ± 7.09 | 80.0 ± 3.32 | 83.6 ± 14.1 |
| CRE (mg/dL) | 0.53 ± 0.04 | 0.58 ± 0.02 | 0.50 ± 0.05 | 0.56 ± 0.05 | 0.51 ± 0.02 | 0.50 ± 0.02 |
| GLU (mg/dL) | 120.6 ± 12.77 | 115.8 ± 7.24 | 119.6 ± 22.55 | 138.3 ± 15.02 | 103.50 ± 13.40 | 101.78 ± 9.13 |
| P (mg/dL) | 5.93 ± 0.60 | 6.63 ± 0.80 | 6.52 ± 1.12 | 6.02 ± 0.74 | 5.47 ± 0.38 | 5.70 ± 0.61 |
| TP (mg/dL) | 7.42 ± 0.21 | 7.54 ± 0.33 | 7.54 ± 0.20 | 7.38 ± 0.25 | 7.34 ± 0.18 | 7.29 ± 0.18 |
| TRG (mg/dL) | 51.82 ± 19.69 | 41.14 ± 3.49 | 41.20 ± 8.06 | 47.72 ± 9.48 | 70.20 ± 7.85 | 61.38 ± 16.01 |
| Urea (mg/dL) | 41.60 ± 8.25 | 44.40 ± 4.51 | 40.50 ± 6.48 | 45.44 ± 4.77 | 39.32 ± 2.27 | 39.42 ± 5.83 |
| AST (U/L) | 169.34 ± 20.92 | 170.22 ± 17.26 | 174.40 ± 16.35 | 164.12 ± 18.08 | 157.22 ± 13.46 | 180.42 ± 37.22 |
| TBL (mg/dL) | 0.10 ± 0.01 | 0.11 ± 0.03 | 0.10 ± 0.02 | 0.09 ± 0.01 | 0.11 ± 0.02 | 0.12 ± 0.01 |
| ALT (U/L) | 42.98 ± 2.62 | 43.62 ± 6.11 | 44.00 ± 5.02 | 42.60 ± 3.17 | 48.66 ± 4.26 | 48.40 ± 7.53 |
| Na (mmol/L) | 142.14 ± 1.88 | 144.18 ± 0.65 | 142.96 ± 0.58 | 143.84 ± 0.55 | 139.00 ± 0.51 | 139.64 ± 0.97 |
| K (mmol/L) | 5.34 ± 0.12 | 4.89 ± 0.10** | 5.12 ± 0.31 | 5.27 ± 0.10 | 5.07 ± 0.28 | 4.99 ± 0.34 |

F: Female;
SD: Standard Deviation;
R: Recovery;
n: Number of Animals;
ALB: Albumin;
ALP: Alkaline Phosphatase;
Ca: Calcium;
CHL: Cholesterol;
CRE: Creatinine;
GLU: Glucose;
P: Phosphorus;
TP: Total Protein;
TRG: Triglycerides;
AST: Aspartate Aminotransferase;
TBL: Total Bilirubin;
ALT: Alanine Aminotransferase;
Na: Sodium;
K: Potassium;
*p<0.05 and **p<0.01 vs G1.

TABLE 17
Summary of urinalysis parameters of male rats

| | Group, sex & Dose (mg/kg b.w.) | | | | | |
|---|---|---|---|---|---|---|
| | | | | G4, M & | | G4R, M & |
| Parameter | G1, M & 0 | G2, M & 250 | G3, M & 500 | 1000 | G1R, M & 0 | 1000 |
| Specific | 1.000 ± 0.000 | 1.000 ± 0.000 | 1.000 ± 0.000 | 1.000 ± 0.000 | 1.000 ± 0.000 | 1.003 ± 0.004 |
| gravity | | | | | | |
| pH | 8.40 ± 0.55 | 8.20 ± 0.45 | 8.80 ± 0.45 | 8.40 ± 0.55 | 8.00 ± 0.00 | 8.00 ± 0.71 |
| Volume (mL) | 2.58 ± 0.50 | 2.54 ± 0.30 | 2.72 ± 0.37 | 2.44 ± 0.38 | 2.12 ± 0.13 | 2.14 ± 0.15 |

Observation/Number of animals showing observation

| | | | | | | |
|---|---|---|---|---|---|---|
| Colour | Clear/5 | Clear/5 | Clear/5 | Clear/5 | Clear/5 | Clear/5 |
| Appearance | Yellow/5 | Yellow/5 | Yellow/5 | Yellow/5 | Yellow/5 | Yellow/5 |
| Alb (mg/dL) | 30/1, | 30/1, | 500/3, | 100/2, | neg/5 | 100/1, |
| | 500/1, | 100/1, | neg/2 | neg/3 | | 500/1, |
| | neg/3 | neg/3 | | | | neg/3 |
| Glucose | neg/5 | neg/5 | neg/5 | neg/5 | neg/5 | neg/5 |
| (mg/dL) | | | | | | |
| Occult blood | neg/5 | neg/5 | neg/5 | neg/5 | 5-10/1 | neg/5 |
| (Ery/μL) | | | | | neg/4 | |

M: Male;
R: Recovery;
n: No. of Animals;
SD: Standard deviation;
Alb: Albumin;
neg: Negative

TABLE 18
Summary of urinalysis parameters of female rats

| | Group, sex & Dose (mg/kg b.w.) | | | | | |
|---|---|---|---|---|---|---|
| | | | | | | G4R, F & |
| Parameter | G1, F & 0 | G2, F & 250 | G3, F & 500 | G4, F & 1000 | G1R, F & 0 | 1000 |
| Specific | 1.000 ± 0.000 | 1.000 ± 0.000 | 1.000 ± 0.000 | 1.000 ± 0.000 | 1.000 ± 0.000 | 1.003 ± 0.004 |
| gravity | | | | | | |
| pH | 8.40 ± 0.55 | 8.80 ± 0.45 | 8.80 ± 0.45 | 8.60 ± 0.55 | 8.00 ± 0.00 | 8.00 ± 0.00 |
| Volume (mL) | 2.60 ± 0.51 | 2.58 ± 0.53 | 2.64 ± 0.48 | 2.64 ± 0.57 | 2.90 ± 0.74 | 2.80 ± 0.57 |

Observation/Number of animals showing observation

| | | | | | | |
|---|---|---|---|---|---|---|
| Colour | Clear/5 | Clear/5 | Clear/5 | Clear/5 | Clear/5 | Clear/5 |
| Appearance | Yellow/5 | Yellow/5 | Yellow/5 | Yellow/5 | Yellow/5 | Yellow/5 |
| Alb (mg/dL) | 500/3, | 100/1 | 100/1 | 100/1 | 30/2, | 30/1, |
| | neg/2 | 500/3, | 500/3, | 500/2, | 500/2, | 500/1, |
| | | neg/1 | neg/1 | neg/2 | neg/1 | neg/3 |
| Glucose | neg/5 | neg/5 | neg/5 | neg/5 | neg/5 | neg/5 |
| (mg/dL) | | | | | | |
| Occult blood | neg/5 | neg/5 | neg/5 | neg/5 | neg/5 | neg/5 |
| (Ery/μL) | | | | | | |

F: Female;
R: Recovery;
n: No. of Animals;
SD: Standard deviation;
Alb: Albumin;
neg: Negative

Both absolute (Table 19 & 20) and relative organ weights (Table 21 & 22) did not reveal any treatment-related effects in any of the treatment groups.

TABLE 19
Summary of organ weight of male rats

| | Group, sex & Dose (mg/kg b.w.) | | | | | |
|---|---|---|---|---|---|---|
| | | | | G4, M & | | G4R, M & |
| Parameter | G1, M & 0 | G2, M & 250 | G3, M & 500 | 1000 | G1R, M & 0 | 1000 |
| Adrenals (g) | 0.06 ± 0.005 | 0.06 ± 0.013 | 0.05 ± 0.006 | 0.05 ± 0.011 | 0.06 ± 0.013 | 0.06 ± 0.011 |
| Brain (g) | 1.68 ± 0.174 | 1.71 ± 0.132 | 1.77 ± 0.049 | 1.64 ± 0.187 | 1.60 ± 0.221 | 1.59 ± 0.112 |
| Heart (g) | 0.79 ± 0.096 | 0.83 ± 0.109 | 0.81 ± 0.093 | 0.77 ± 0.153 | 0.92 ± 0.073 | 0.97 ± 0.138 |
| Kidneys (g) | 1.85 ± 0.184 | 2.02 ± 0.118 | 1.87 ± 0.195 | 1.73 ± 0.219 | 1.81 ± 0.172 | 2.18 ± 0.261# |
| Liver (g) | 8.45 ± 0.970 | 9.65 ± 0.952 | 9.23 ± 0.856 | 9.40 ± 1.182 | 8.74 ± 0.851 | 10.41 ± 2.10 |
| Spleen (g) | 0.74 ± 0.225 | 1.02 ± 0.100* | 0.88 ± 0.151 | 0.88 ± 0.088 | 0.70 ± 0.212 | 0.80 ± 0.150 |
| Thymus (g) | 0.32 ± 0.043 | 0.265 ± 0.105 | 0.29 ± 0.059 | 0.27 ± 0.054 | 0.29 ± 0.108 | 0.36 ± 0.062 |
| Testes (g) | 2.51 ± 0.441 | 2.46 ± 0.118 | 2.55 ± 0.190 | 2.54 ± 0.246 | 2.77 ± 0.348 | 2.72 ± 0.092 |
| Epididymis | 0.83 ± 0.086 | 0.83 ± 0.157 | 0.77 ± 0.060 | 0.86 ± 0.068 | 1.10 ± 0.100 | 1.05 ± 0.100 |
| Prostate, seminal | 1.35 ± 0.333 | 1.05 ± 0.306 | 1.04 ± 0.184 | 1.46 ± 0.228 | 1.48 ± 0.262 | 1.22 ± 0.380 |
| vesicles with | | | | | | |
| coagulating | | | | | | |
| glands (g) | | | | | | |

M: Male; R: Recovery; SD: Standard Deviation; n: No. of Animals; * p<0.05 vs G1; #p<0.05 vs G1R

TABLE 20
Summary of organ weight of female rats

| | Group, sex & Dose (mg/kg b.w.) | | | | | |
|---|---|---|---|---|---|---|
| | | | | | | G4R, F & |
| Parameter | G1, F & 0 | G2, F & 250 | G3, F & 500 | G4, F & 1000 | G1R, F & 0 | 1000 |
| Adrenals (g) | 0.07 ± 0.016 | 0.07 ± 0.009 | 0.071 ± 0.012 | 0.08 ± 0.014 | 0.07 ± 0.013 | 0.08 ± 0.002 |
| Brain (g) | 1.65 ± 0.061 | 1.63 ± 0.049 | 1.63 ± 0.186 | 1.69 ± 0.093 | 1.55 ± 0.161 | 1.68 ± 0.186 |
| Heart (g) | 0.62 ± 0.074 | 0.64 ± 0.104 | 0.72 ± 0.129 | 0.68 ± 0.028 | 0.78 ± 0.123 | 0.83 ± 0.140 |
| Kidneys (g) | 1.37 ± 0.230 | 1.30 ± 0.167 | 1.49 ± 0.266 | 1.51 ± 0.204 | 1.52 ± 0.130 | 1.54 ± 0.143 |
| Liver (g) | 6.78 ± 1.034 | 7.29 ± 1.012 | 7.80 ± 1.444 | 7.40 ± 0.687 | 7.606 ± 0.927 | 8.01 ± 0.599 |
| Spleen (g) | 0.58 ± 0.187 | 0.59 ± 0.201 | 0.56 ± 0.080 | 0.56 ± 0.054 | 0.69 ± 0.106 | 0.64 ± 0.113 |
| Thymus (g) | 0.28 ± 0.091 | 0.30 ± 0.055 | 0.40 ± 0.170 | 0.43 ± 0.054 | 0.34 ± 0.063 | 0.28 ± 0.038 |
| Ovaries (g) | 0.09 ± 0.017 | 0.10 ± 0.013 | 0.09 ± 0.020 | 0.10 ± 0.025 | 0.10 ± 0.011 | 0.11 ± 0.011 |
| Uterus along | 0.51 ± 0.165 | 0.52 ± 0.141 | 0.50 ± 0.123 | 0.67 ± 0.120 | 0.59 ± 0.140 | 0.65 ± 0.183 |
| with vagina and | | | | | | |
| cervix | | | | | | |

F: Female;
R: Recovery;
SD: Standard Deviation;
n: No. of Animals

TABLE 21
Summary of relative organ weight of male rats

| | Group, sex & Dose (mg/kg b.w.) | | | | | |
|---|---|---|---|---|---|---|
| | | | | G4, M & | | G4R, M & |
| Parameter | G1, M & 0 | G2, M & 250 | G3, M & 500 | 1000 | G1R, M & 0 | 1000 |
| Fasting body | 222.02 ± 21.47 | 220.33 ± 24.03 | 209.07 ± 16.25 | 210.86 ± 27.41 | 216.34 ± 16.55 | 248.96 ± 38.71 |
| weight (g) | | | | | | |
| Adrenals (g) | 0.03 ± 0.004 | 0.03 ± 0.004 | 0.02 ± 0.004 | 0.02 ± 0.005 | 0.03 ± 0.004 | 0.02 ± 0.005 |
| Brain (g) | 0.76 ± 0.080 | 0.78 ± 0.069 | 0.85 ± 0.059 | 0.79 ± 0.142 | 0.74 ± 0.092 | 0.65 ± 0.101 |
| Heart (g) | 0.36 ± 0.049 | 0.38 ± 0.081 | 0.39 ± 0.033 | 0.36 ± 0.031 | 0.42 ± 0.016 | 0.39 ± 0.066 |
| Kidneys (g) | 0.83 ± 0.062 | 0.92 ± 0.077 | 0.90 ± 0.041 | 0.82 ± 0.015 | 0.84 ± 0.041 | 0.89 ± 0.133 |
| Liver (g) | 3.82 ± 0.419 | 4.43 ± 0.745 | 4.44 ± 0.552 | 4.47 ± 0.369 | 4.04 ± 0.234 | 4.24 ± 0.993 |
| Spleen (g) | 0.33 ± 0.104 | 0.47 ± 0.053* | 0.42 ± 0.055 | 0.43 ± 0.072 | 0.33 ± 0.107 | 0.32 ± 0.069 |
| Thymus (g) | 0.14 ± 0.019 | 0.12 ± 0.058 | 0.14 ± 0.033 | 0.13 ± 0.022 | 0.14 ± 0.053 | 0.15 ± 0.038 |
| Testes (g) | 1.14 ± 0.197 | 1.13 ± 0.157 | 1.22 ± 0.020 | 1.21 ± 0.162 | 1.28 ± 0.191 | 1.11 ± 0.176 |
| Epididymis | 0.38 ± 0.056 | 0.38 ± 0.098 | 0.37 ± 0.028 | 0.41 ± 0.059 | 0.51 ± 0.066 | 0.43 ± 0.055 |
| Prostate, seminal | 0.61 ± 0.119 | 0.48 ± 0.150 | 0.50 ± 0.058 | 0.69 ± 0.054 | 0.69 ± 0.124 | 0.49 ± 0.130# |
| vesicles with | | | | | | |
| coagulating | | | | | | |
| glands (g) | | | | | | |

M: Male;
R: Recovery;
SD: Standard Deviation;
n: No. of Animals;
*p<0.05 vs G1;
#p<0.05 vs G1R

TABLE 22
Summary of relative organ weight of female rats

| | Group, sex & Dose (mg/kg b.w.) | | | | | |
|---|---|---|---|---|---|---|
| | | | | | | G4R, F & |
| Parameter | G1, F & 0 | G2, F & 250 | G3, F & 500 | G4, F & 1000 | G1R, F & 0 | 1000 |
| Fasting body | 160.65 ± 19.95 | 168.98 ± 21.62 | 178.82 ± 25.22 | 167.77 ± 14.90 | 178.91 ± 7.09 | 193.56 ± 22.45 |
| weight (g) | | | | | | |
| Adrenals (g) | 0.04 ± 0.013 | 0.04 ± 0.006 | 0.04 ± 0.003 | 0.05 ± 0.008 | 0.04 ± 0.007 | 0.04 ± 0.005 |
| Brain (g) | 1.04 ± 0.171 | 0.98 ± 0.128 | 0.92 ± 0.066 | 1.01 ± 0.059 | 0.87 ± 0.121 | 0.87 ± 0.032 |
| Heart (g) | 0.39 ± 0.073 | 0.38 ± 0.042 | 0.40 ± 0.057 | 0.41 ± 0.022 | 0.44 ± 0.058 | 0.43 ± 0.054 |
| Kidneys (g) | 0.87 ± 0.214 | 0.77 ± 0.102 | 0.83 ± 0.065 | 0.90 ± 0.118 | 0.85 ± 0.055 | 0.80 ± 0.026 |
| Liver (g) | 4.31 ± 1.053 | 4.34 ± 0.557 | 4.35 ± 0.271 | 4.41 ± 0.137 | 4.25 ± 0.459 | 4.16 ± 0.306 |
| Spleen (g) | 0.38 ± 0.166 | 0.35 ± 0.124 | 0.32 ± 0.054 | 0.33 ± 0.053 | 0.39 ± 0.047 | 0.33 ± 0.062 |
| Thymus (g) | 0.18 ± 0.076 | 0.18 ± 0.015 | 0.22 ± 0.067 | 0.26 ± 0.048 | 0.19 ± 0.032 | 0.15 ± 0.034 |
| Ovaries (g) | 0.06 ± 0.010 | 0.06 ± 0.003 | 0.05 ± 0.009 | 0.06 ± 0.014 | 0.06 ± 0.006 | 0.06 ± 0.006 |
| Uterus along | 0.32 ± 0.110 | 0.31 ± 0.103 | 0.28 ± 0.084 | 0.40 ± 0.085 | 0.34 ± 0.086 | 0.33 ± 0.073 |
| with vagina and | | | | | | |
| cervix | | | | | | |

F: Female;
R: Recovery;
SD: Standard Deviation;
n: No. of Animals

No treatment related gross and/or histopathological findings were observed in any of organ/tissues which are examined in both sexes (Table 23 & 24).

TABLE 23
Summary of histopathology findings of male rats

| | Group, Sex & Dose | |
|---|---|---|
| | Observation/ Number of Animals showing that observation | |
| | G1, Male & | G4, Male & |
| Organs | 0 mg/kg b. w. | 1000 mg/kg b. w. |
| Brain | NAD/5 | NAD/5 |
| Spinal cord | NAD/5 | NAD/5 |
| Eyes with optic nerve | NAD/5 | NAD/5 |
| Trachea | NAD/5 | NAD/5 |
| Thyroid & Parathyroid | NAD/5 | NAD/5 |
| Spleen | NAD/5 | NAD/5 |
| Thymus | NAD/5 | NAD/5 |
| Adrenals | NAD/5 | NAD/5 |
| Lungs | Alveolar hemorrhages/ | Thickening of alveolar |
| | 1; Alveolar wall | wall/3 NAD/2 |
| | thickening/1; NAD/3 | |
| Heart | NAD/5 | NAD/5 |
| Stomach | NAD/5 | NAD/5 |
| Small Intestine (Duodenum, | Sub mucosal lymphoid tissue | Sub mucosal lymphoid tissue |
| jejunum, terminal Ileum | hyperplasia in ileum/ | hyperplasia in ileum/1; |
| with peyer's patches ) | 1; NAD/4 | NAD/4 |
| Large Intestine | Sub mucosal lymphoid tissue | Sub mucosal lymphoid tissue |
| (colon, rectum,c ecum) | hyperplasia in colon/1; | hyperplasia in colon/1; |
| | NAD/4 | NAD/4 |
| Liver | Sinusoidal hemorrhages/1; | Peri portal infiltration |
| | Peri portal infiltration of | of inflammatory |
| | inflammatory cells/1; NAD/3 | cells/2; NAD/3 |
| Kidneys | Foci of tubular infiltration of | NAD/5 |
| | inflammatory cells/1; NAD/4 | |
| Urinary bladder | NAD/5 | NAD/5 |
| Epididymis | NAD/5 | NAD/5 |
| Prostate, Seminal vesicles | Mucosal degeneration of | Mucosal degeneration of |
| with coagulating glands | epithelial cells/1, NAD/4 | epithelial cells/1, NAD/4 |
| Testes | NAD/5 | NAD/5 |
| Sciatic nerve | NAD/5 | NAD/5 |
| Mesenteric lymph node | NAD/5 | NAD/5 |
| Femur bone | NAD/5 | NAD/5 |
| Skeletal muscle | NAD/5 | NAD/5 |

NAD: No Abnormality Detected

TABLE 24
Summary of histopathology findings of female rats

| | Group, Sex & Dose | |
|---|---|---|
| | Observation/ Number of Animals showing that observation | |
| | G1, Female & | G4, Female & |
| Organs | 0 mg/kg b. w. | 1000 mg/kg b. w |
| Brain | Foci of cerebral necrosis/ | NAD/5 |
| | 1; NAD/4 | |
| Spinal cord | NAD/5 | NAD/5 |
| Eyes with optic nerve | NAD/5 | NAD/5 |
| Trachea | NAD/5 | NAD/5 |
| Thyroid & parathyroid | NAD/5 | NAD/5 |
| Spleen | NAD/5 | NAD/5 |
| Thymus | NAD/5 | NAD/5 |
| Adrenals | NAD/5 | NAD/5 |
| Lungs | Alveolar hemorrhages/1; | Thickening of alveolar wall/ |
| | Thickening of alveolar | 2; NAD/3 |
| | wall/2; NAD/2 | |
| Heart | NAD/5 | NAD/5 |
| Stomach | NAD/5 | NAD/5 |
| Small Intestine | Sub mucosal lymphoid tissue | Sub mucosal lymphoid tissue |
| (Duodenum, jejunum, | hyperplasia in ileum/1; | hyperplasia in ileum/1; |
| termina Ileum | | NAD/4 |
| with peyer's patches) | | |
| | NAD/4 | |
| Large Intestine | NAD/5 | Sub mucosal lymphoid tissue |
| (colon, rectum, cecum) | | hyperplasia in colon/2; NAD/3 |
| Liver | Sinusoidal hemorrhages/1; | Sinusoidal hemorrhages/2; |
| | Peri portal infiltration of | NAD/3 |
| | inflammatory cells/1; NAD/3 | |
| Kidneys | Tubular hemorrhages/1; | Tubular infiltration of |
| | Tubular degeneration/1 | inflammatory cells/2; NAD/3 |
| | NAD/4 | |
| Urinary bladder | NAD/5 | NAD/5 |
| Uterus along with | NAD/5 | NAD/5 |
| vagina and cervix | | |
| Ovaries | NAD/5 | NAD/5 |
| Mammary glands | NAD/5 | NAD/5 |
| Sciatic nerve | NAD/5 | NAD/5 |
| Mesenteric lymph node | NAD/5 | NAD/5 |
| Femur bone | NAD/5 | NAD/5 |
| Skeletal muscle | NAD/5 | NAD/5 |

NAD: No Abnormality Detected

The results demonstrated that sample extract did not reveal any toxicological effects under the test conditions and the “No Observed Adverse Effect Level (NOAEL)” was found to be 1000 mg/kg b.w. following repeated 28-day oral route administration followed by 14 days recovery period in Wistar rats.

### REFERENCES

1) Aditya N P, Chimote G, Gunalan K, et al. (2012) Curcuminoids-loaded liposomes in combination with arteether protects against Plasmodium berghei infection in mice. Exp Parasitol 131:292-299.
2) Amalraj A, Pius A, Gopi S, Gopi S (2017) Biological activities of curcuminoids, other biomolecules from turmeric and their derivatives—a review. J Tradit Complement Med 7(2):205-33.
3) Anand P, Kunnumakkara A B, Newman R A, et al. (2007) Bioavailability of curcumin: problems and promises. Mol Pharm 4(6):807-18.
4) Anand P, Sundaram C, Jhurani S, et al. (2008) Curcumin and cancer: an “old-age” disease with an “age-old” solution. Cancer Lett 267(1):133-64.
5) Bajda M, Więckowska A, Hebda M, et al. (2013) Structure-based search for new inhibitors of cholinesterases. Int J Mol Sci 14(3):5608-5632.
6) Cuomo F, Cofelice M, Venditti F, et al. (2018) In-vitro digestion of curcumin loaded chitosan-coated liposomes. Colloids Surf B Biointerfaces 168:29-34.
7) Ellman G L, Courtney K D, Andres V, (1961) A new and rapid colorimetric determination of acetylcholinesterase activity. Biochem Pharmacol 7:88-95.
8) Hewlings S J, Kalman D S (2017) Curcumin: A review of its' effects on human health. Foods. 6(10):92.
9) Jurenka J S (2009) Anti-inflammatory properties of curcumin, a major constituent of Curcuma longa: a review of preclinical and clinical research. Altern Med Rev 14(2):141-53.
10) Kalpravidh R W, Siritanaratkul N, Insain P, et al. (2010) Improvement in oxidative stress and antioxidant parameters in beta-thalassemia/Hb E patients treated with curcuminoids. Clin Biochem 43:424-429.
11) Kalaycioğlu Z, Gazioğlu I, Erim F B. (2017) Comparison of antioxidant, anticholinesterase, and antidiabetic activities of three curcuminoids isolated from Curcuma longa L. Natural Product Ress 31(24):2914-2917.
12) Kemal C, Louis-Flamberg P, Krupinski-Olsen R, et al. (1987) Reductive inactivation of soybean lipoxygenase 1 by catechols: a possible mechanism for regulation of lipoxygenase activity. Biochemistry 26:7064-7072.
13) Lim H S, Park S H, Ghafoor K, et al. (2011) Quality and antioxidant properties of bread containing turmeric (Curcuma longa L.) cultivated in South Korea. Food Chem. 124:1577-1582.
14) Mosmann T. Rapid colorimetric assay for cellular growth and survival: application to proliferation and cytotoxicity assays, J Immunol Methods 1983; 65:55-63.
15) Noro T, Oda Y, Miyase T, et al. (1983) Inhibitors of xanthine oxidase from the flowers and buds of Daphne genkwa, Chem Pharm Bull (Tokyo) 31:3984-3987.
16) Panahi Y, Saadat A, Beiraghdar F, et al. (2014) Antioxidant effects of bioavailability-enhanced curcuminoids in patients with solid tumors: a randomized double-blind placebo-controlled trial. J Funct Foods 6:615-622.
17) Paramasivam M, Poi R, Banerjee H, et al. (2009) High-performance thin layer chromatographic method for quantitative determination of curcuminoids in Curcuma longa germplasm. Food Chem 113:640-644.
18) Re R, Pellegrini N, Proteggente A, et al. (1999) Antioxidant activity applying an improved ABTS radical cation decolorization assay. Free Radic Biol Med 26:1231-1237.
19) Ryan L, Connell O O, Sullivan O et al. (2008) Micellarisation of carotenoids from raw and cooked vegetables. Plant Foods Hum Nutr 63:127-133.
20 ) Sandur S K, Pandey M K, Sung B, et al. (2007) Curcumin, demethoxycurcumin, bisdemethoxycurcumin, tetrahydrocurcumin and turmerones differentially regulate anti-inflammatory and anti-proliferative responses through a ROS-independent mechanism. Carcinogenesis 28(8):1765-73.
21) Siviero A, Gallo E, Maggini V, et al. (2015) Curcumin, a golden spice with a low bioavailability. J Herb Med 5:57-70.
22) Soler-Rivas C, Espin J C, Wichers H J (2000) An easy and fast test to compare total free radical scavenger capacity of foodstuffs. Phytochem Anal 11:330-338.
23) Sreejayan, Rao M N (1997) Nitric oxide scavenging by curctuninoids. J Pharm Pharmacol 49:105407.

24) Stavrakov G, Philipova I, Zheleva D, et al. (2016) Docking-based design of galantamine derivatives with dual-site binding to acetylcholinesterase. Molecular Informatics
25) Thavorn K, Mamdani M M, Straus S E (2014) Efficacy of turmeric in the treatment of digestive disorders: a systematic review and meta-analysis protocol. Syst Rev 3:71.
26) Venkatachalam U, Muthukrishnan S (2012) Free radical scavenging activity of ethanolic extract of Desmodium gangeticum. J Acute Med 2:36-42.
27) Yodkeeree S, Chaiwangyen W, Garbisa S, et al. (2009) Curcumin, demethoxycurcumin and bisdemethoxycurcumin differentially inhibit cancer cell invasion through the down-regulation of MMPs and uPA. J Nutr Biochem 20(2):87-95.
28) Yu C C, Yang M D, Lin H Y, et al. (2015) Bisdemethoxycurcumin (BDMC) alters gene expression-associated cell cycle, cell migration and invasion and tumor progression in human lung cancer NCI-H460 cells. In Vivo 29(6):711-728.

cm

1

-

40

. (canceled)

41. A method of treating Parkinson's disease, comprising administering to a subject in need thereof an effective amount of a composition comprising a mixture of curcuminoids, wherein said mixture contains an amount of bisdemethoxycurcumin (BDMC) and an amount of curcumin, wherein said amount of BDMC is greater than said amount of curcumin.

42. The method of claim 41, wherein said mixture comprises about 75±5% w/w BDMC and about 1.2±0.8% w/w curcumin.

43. The method of claim 41, wherein said mixture comprises about 62.5 parts BDMC and about 1 part curcumin.

44. The method of claim 41, wherein said mixture further comprises demethoxycurcumin (DMC).

45. The method of claim 44, wherein said mixture comprises about 10±5% w/w DMC.

46. The method of claim 44, wherein said mixture comprises about 62.5 parts BDMC, about 8.3 parts DMC and about 1 part curcumin.

47. The method of claim 41, wherein said mixture is an extract of turmeric.

48. The method of claim 47, wherein said extract is turmeric rhizome extract.

49. The method of claim 41, wherein said composition is in a form selected from a powder, liquid, pill, tablet, pellet, capsule, thin film, solution, spray, syrup, linctus, lozenge, pastille, chewing gum, paste, vapor, suspension, emulsion, ointment, cream, lotion, liniment, gel, drop, topical patch, buccal patch, bead, gummy, gel, sol, injection, and combinations thereof.

50. The method of claim 41, wherein said composition is combined with a food, beverage or nutritional supplement.

51. The method of claim 41, wherein said subject is at risk of developing Parkinson's disease.

52. The method of claim 41, wherein said subject has Parkinson's disease.

53. The method of claim 41, wherein said composition is administered systemically.

54. The method of claim 41, wherein said composition is administered orally.

55. The method of claim 41, wherein said composition is administered at a dose of about 100 mg/kg body weight, about 200 mg/kg body weight, about 250 mg/kg body weight, or about 300 mg/kg body weight.

56. The method of claim 41, wherein administering said composition arrests, inhibits, delays or prevents the progression of the symptoms of Parkinson's disease.
